(19) United States
(12) Patent Application Publication
(10) Pub. No.: US 2011/0212122 A1
(43) Pub. Date: Sep. 1, 2011

(54) Nerve Growth Factor Conjugates and Uses Thereof

(76) Inventors: Martin Bachmann, Seuzach (CH); Gary Jennings, Zurich (CH); Till Röhn, Zurich (CH)

(21) Appl. No.: 12/809,261

(22) PCT Filed: Dec. 22, 2008
(86) PCT No.: PCT/EP2008/068209

§ 371 Date: Mar. 24, 2011

(30) Foreign Application Priority Data
Dec. 20, 2007 (EP) 07150234.8

(51) Int. Cl. A61K 39/12; C07K 14/48; A61P 19/02; A61P 25/00

(52) U.S. Cl. 424/192.1; 530/399

(57) ABSTRACT

The present invention is in the fields of medicine, public health, immunology, molecular biology and virology. The invention provides composition comprising a virus-like particle (VLP) linked to at least one antigen, wherein said antigen is NGF antigen. The invention also provides a process for producing the composition. The compositions of this invention are useful in the production of vaccines, in particular, for the treatment of pain. Moreover, the compositions of the invention induce efficient immune responses, in particular antibody responses.

### FIELD OF THE INVENTION

The present invention is in the fields of medicine, public health, immunology, molecular biology and virology. The invention provides composition comprising a virus-like particle (VLP) linked to at least one antigen, wherein said antigen is a NGF antigen. The invention also provides a process for producing the composition. The compositions of this invention are useful in the production of vaccines, in particular, for the treatment of chronic pain. Moreover, the compositions of the invention induce efficient immune responses, in particular antibody responses.

### RELATED ART

Nerve growth factor (NGF), which is also known as NGFβ, is a neurotrophic factor and the founding member of the family of neurotrophins which consists of Brain Derived Neurotropic Factor (BDNF), neurotrophin 3 and neurotrophin 4/5 besides NGF (Pezet and McMahon, Annu. Rev. Neurosci. 29: 507-38 (2006)). Like all neurotrophins, NGF is expressed as a pro-protein of approximately 27 kDa and it is cleaved to a mature form of approximately 14 kDa (Edwards et al., J Biol Chem. 263(14): 6810-5(1988); Seidah et al., Biochem J. 314: 951-60 (1996)). This mature form exerts its biological function by binding to two receptors: the common receptor p75^{NTR}, which binds to all neurotrophins with low affinity, and to the high affinity tropomyosine receptor kinase A (trkA) receptor. TrkA is a receptor tyrosine kinase which dimerizes upon ligand binding and activates different signaling pathways (Patapontian and Reichardt, Curr Opin Neurobiol. 11(3): 272-80 2001)). During early development these signaling pathways activated by NGF-trkA interaction block apoptosis and promote the survival and neural outgrowth of sensory neurons of the nociceptive system (Patel et al., Neuron 25(2): 345-57 (2000)). After birth these neurons loose their dependence on NGF for survival but NGF continues to exert profound biological effects on nociceptors also during the postnatal period. It regulates the expression of neurotransmitters, receptors and voltage-gated ion channels thereby controlling the responsiveness of nociceptors. Another functionally important action of NGF is to sensitize nociceptor responses through post translational controls which lead to enhanced responsiveness of transient receptor potential vanilloid 1 (TRPV1) ion channels (Pezet and McMahon, Annu Rev. Neurosci. 29: 507-38 (2006)).

NGF is involved in the mediation of pain perception in animals and humans due to its role in the regulation of nociceptor responsiveness in adults. Small subcutaneous or intramuscular injections of NGF give rise to pain and tenderness lasting for days (Pezet and McMahon, Annu Rev. Neurosci. 29: 507-38 (2006)). In humans a correlation between NGF levels and pain intensity could be assessed in a number of painful conditions like chronic prostatitis (Miller et al., Urology, 2002. 59(4): 603-8 (2002)) interstitial cystitis (Lowe et al., Br J Urol. 79(4): 572-7 (1997)) and others. Essentially, two types of pain can be distinguished: Nociceptive pain and neuropathic pain. While nociceptive pain arises from the stimulation of nociceptors caused by tissue damage for example in the course of an inflammation, after injury or surgical incision, neuropathic pain originates from pathology of the nervous system itself for example after nerve compression or trauma or after an infection or autoimmunity affecting sensory neurons.

During an inflammation NGF acts as an algogenic inflammatory mediator. It is produced by a number of different cell types in the periphery like keratinocytes, epithelial cells, smooth muscle cells and Schwann cells and during an inflammation especially by mast cells and macrophages. NGF levels are substantially elevated during inflammation and chronic painful conditions in animals and humans. Blocking of NGF has been shown to significantly decrease pain sensitivity in a number of nociceptive pain models in rodents For example it decreased thermal and mechanical hyperalgesia after cutaneous injection of CFA (Woolf et al., Br. J. Pharmacol. 121(3):417-24 (1997) and suppressed arthritis associated pain (Shelton et al., Pain 116(1-2): 8-16 (2005)) and bone cancer pain (Sevcik et al., Pain 115(1-2): 128-41(2005)) among others. Although the role of NGF in neuropathic pain is less well established than in inflammatory pain, some groups have also shown the success of anti-NGF treatment in models of neuropathic pain in rodents (Ramer et al., Neurosci. Lett. 251(1): 53-6 (1998), Ramer et al., Eur. J. Neurosci. 11(3): 837-46 (1999)).

U.S. Pat. No. 5,147,294 claims the use of NGF antagonists—among them NGF monoclonal antibodies—for the treatment of pain. Since then a number of patents have been filed which claim the use of monoclonal antibodies against NGF for the treatment of chronic pain.

Chronic pain is a serious health problem affecting approximately 20% of the European and U.S. population. Less than 30% of patients suffering from chronic pain obtain adequate relief with current therapies (Pezet and McMahon, Annu Rev. Neurosci. 29: 507-38 (2006). Even more, existing drugs used for the treatment of chronic pain, mostly opioids and non-steroidal anti-inflammatory drugs (NSAIDs), are accompanied by serious side effects if taken for an extended period of time.

### SUMMARY OF THE INVENTION

The novel therapeutic strategy which is disclosed herein is based on active immunization against NGFβ. Disclosed are compositions which induce the production of NGF-neutralizing antibodies by the immune system of a patient. Active immunization may result in enduring neutralization of NGFβ, and, thus, may reduce the need for daily drug intake. We have, now, surprisingly found that the inventive compositions and vaccines, respectively, comprising at least one NGF antigen, are capable of inducing immune responses, in particular antibody responses, leading to high antibody titer against NGF. Moreover, we have surprisingly found that inventive compositions and vaccines, respectively, comprising at least one NGF antigen, are capable of reducing pain in animal models for chronic pain. This indicates that the immune responses, in particular the antibodies generated by the inventive compositions and vaccines, respectively, are, thus, capable of specifically binding NGF in vivo, and neutralizing and inhibiting its function.

One aspect of the invention is therefore a composition comprising: (a) a virus-like particle (VLP) with at least one first attachment site; and (b) at least one antigen with at least one second attachment site, wherein said at least one antigen is a NGF antigen; and wherein (a) and (b) are linked through said at least one first and said at least one second attachment site. NGFβ which is a receptor ligand comprising biologic activity which may result in undesired side effects when administered to an animal. The invention thus provides compositions wherein said NGF protein is an NGF mutein, and wherein said NGF mutein comprised reduced or no biologic activity while it still retains its capability of inducing NGFβ neutralizing antibodies. Thus, in a preferred embodiment of the invention, said NGF antigen is an NGF mutein.

Generally, highly undesirable side effects may be associated to the induction of T cell responses by anti NGFβ vaccines. The compositions of the invention however are particularly useful to efficiently induce strong antibody responses against the NGF antigen within the indicated context while lowering or eliminating unwanted T cell responses. In a preferred embodiment the composition of the invention therefore further comprises at least one polyanionic macromolecule, wherein said polyanionic macromolecule is packaged into said VLP, and wherein preferably said polyanionic macromolecule is a polyanionic polypeptide, and wherein preferably said polyanionic polypeptide is polyglutamic acid or polyaspartic acid, most preferably polyglutamic acid.

A further aspect of the invention is a vaccine composition comprising a therapeutically effective amount of the composition of the invention.

A further aspect of the invention is a pharmaceutical composition comprising: (a) a composition of the invention; and (b) a pharmaceutically acceptable carrier.

A further aspect of the invention is method of immunization, preferably against NGFβ, said method comprising administering the composition of the invention, the vaccine composition of the invention, or the pharmaceutical composition of the invention to an animal, preferably to a human.

A further aspect of the invention is the composition of the invention, the vaccine composition of the invention, or the pharmaceutical composition of the invention for use as a medicament.

A further aspect of the invention is the use of the composition of the invention, or the use of the vaccine composition of the invention for the manufacture of a medicament for the treatment of pain, preferably of chronic pain.

A further aspect of the invention is composition of the invention, or a vaccine composition of the invention, or a pharmaceutical composition of the invention for use in the treatment of pain, preferably of chronic pain.

A further aspect of the invention is a method of producing the composition of the invention, said method comprising: (a) providing a VLP with at least one first attachment site; (b) providing at least one NGF antigen with at least one second attachment site; and (c) linking said VLP and said NGF antigen to produce said composition, wherein said NGF antigen and said VLP are linked through said at least one first and said at least one second attachment site.

### BRIEF DESCRIPTION OF THE DRAWINGS

FIG. 1: In vitro neutralization of mNGFβ by purified total IgGs of immunized mice. Proliferation of TF-1 cells in response to mNGFβ was determined via BrdU incorporation. Proliferation in response to 10 ng/ml mNGFβ in the presence of 10 μg/ml total IgGs of Qβ immunized animals was set as 100% and the TF-1 proliferation in response to all other conditions calculated in relation to that value. Circles represent values derived after pre-incubation with IgGs from Qβ-immunized animals, squares values derived after pre-incubation with IgGs from Qβ-mNGFβ-His-GGC-immunized animals. Averages of triplicates+/−SD are given.

FIG. 2: Development of clinical scores and average body weight in CIA mice. Mice were immunized at day 0, 10 and 20 and CIA induced at day 27 and 48. A) Clinical arthritic scores of mice immunized with Qβ (filled squares) or Qβ-mNGFβ-His-GGC (open circles). Values are given as average scores of all four limbs per mouse and group (n=8)+/−SEMs. B) Average body weight of mice immunized with Qβ (filled squares) or Qβ-mNGFβ-His-GGC (open circles). Values are given as average body weight (n=8)+/−SEMs.

FIG. 3: Thermal hypersensitivity and mechanical allodynia in zymosan A induced inflammatory pain. Mice were immunized at day 0, 10 and 20 and inflammatory pain induced at day 31 by injection of zymosan A into the left hind foot paw. A) Thermal hypersensitivity of mice immunized with Qβ (filled squares) or Qβ-mNGFβ-His-GGC (open circles) determined as paw withdrawal time. Values given as averages (n=4)+/−SEMs. B) Mechanical allodynia of mice immunized with Qβ (filled squares) or Qβ-mNGFβ-His-GGC (open circles) determined as applied pressure causing paw withdrawal. Values given as averages (n=4)+/−SEMs.

FIG. 4: Thermal hypersensitivity and mechanical allodynia in Zymosan A induced inflammatory pain in Qβ-(PolyGlu)-mNGFβ-His-GGC immunized mice. Mice were immunized at day 0, 14, 28 and 42 and inflammatory pain was induced at day 62 by injection of zymosan A into the left hind foot paw. A) Thermal hypersensitivity of mice immunized with Qβ (PolyGlu) (filled squares) Qβ (PolyGlu)-mNGFβ-His-GGC (filled triangles) or Qβ-mNGFβ-His-GGC (open circles) determined as paw withdrawal time. Values given as averages (n=4)+/−SEMs. B) Mechanical allodynia of mice immunized with Qβ (PolyGlu) (filled squares) Qβ (PolyGlu)-mNGFβ-His-GGC (grey filled circles) or Qβ-mNGFβ-His-GGC (open circles) determined as applied force causing paw withdrawal. Values given as averages (n=4)+/−SEMs.

### DETAILED DESCRIPTION OF THE INVENTION

Unless defined otherwise, all technical and scientific terms used herein have the same meanings as commonly understood by one of ordinary skill in the art to which this invention belongs.

Antigen: As used herein, the term “antigen” refers to a molecule capable of being bound by an antibody or a T cell receptor (TCR) if presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and is given in adjuvant. An antigen can have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens. In a very preferred embodiment, the antigen is an NGF antigen.

Epitope: The term “epitope” refers to continuous or discontinuous portions of a polypeptide, which can be bound immunospecifically by an antibody or by a T-cell receptor within the context of an MHC molecule. Immunospecific binding excludes non-specific binding but does not necessarily exclude cross-reactivity. An epitope typically comprise 5-10 amino acids in a spatial conformation which is unique to the epitope.

Associated: The term “associated” (or its noun association) as used herein refers to all possible ways, preferably chemical interactions, by which two molecules are joined together. Chemical interactions include covalent and non-covalent interactions. Typical examples for non-covalent interactions are ionic interactions, hydrophobic interactions or hydrogen bonds, whereas covalent interactions are based, by way of example, on covalent bonds such as ester, ether, phosphoester, amide, peptide, carbon-phosphorus bonds, carbon-sulfur bonds such as thioether, or imide bonds.

Attachment Site, First: As used herein, the phrase “first attachment site” refers to an element which is naturally occurring with the VLP, preferably with the VLP of an RNA-bacteriophage, or which is artificially added to the VLP preferably to the VLP of an RNA-bacteriophage, and to which the second attachment site may be linked. The first attachment site may be a protein, a polypeptide, an amino acid, a peptide, a sugar, a polynucleotide, a natural or synthetic polymer, a secondary metabolite or compound (biotin, fluorescein, retinol, digoxigenin, metal ions, phenylmethylsulfonylfluoride), or a chemically reactive group such as an amino group, a carboxyl group, a sulfhydryl group, a hydroxyl group, a guanidinyl group, histidinyl group, or a combination thereof. A preferred embodiment of a chemically reactive group being the first attachment site is the amino group of an amino acid such as lysine. In a preferred embodiment said first attachment site is the amino group of a lysine residue, wherein preferably said lysine residue is a lysine residue which is naturally occurring with said VLP, preferably with said VLP of an RNA-bacteriophage.

The first attachment site is located, typically on the surface, and preferably on the outer surface of the VLP, preferably of the VLP of an RNA-bacteriophage, most preferably of an RNA-bacteriophage Qβ. Multiple first attachment sites are present on the surface, preferably on the outer surface, of the virus-like particle, preferably of the VLP of an RNA-bacteriophage, most preferably of the VLP of RNA-bacteriophage Qβ, typically and preferably in a repetitive configuration. In a preferred embodiment the first attachment site is associated with the VLP through at least one covalent bond, preferably through at least one peptide bond. In a further preferred embodiment the first attachment site is naturally occurring with the VLP. Alternatively, in another preferred embodiment the first attachment site is artificially added to the VLP. In a preferred embodiment the first attachment site is associated with said VLP through at least one covalent bond, preferably through at least one peptide bond, wherein said VLP is a VPL of an RNA-bacteriophage, preferably of RNA-bacteriophage Qβ. In a further preferred embodiment said first attachment site is the amino group of a lysine residue, wherein said lysine residue is a lysine residue of a coat protein, preferably of a coat protein of an RNA-bacteriophage, most preferably of RNA-bacteriophage Qβ. In a further preferred embodiment said first attachment site is an amino group of a lysine residue of a coat protein of an RNA-bacteriophage, wherein preferably said coat protein comprises or preferably consists of the amino acid sequence of SEQ ID NO:1. In a further preferred embodiment said first attachment site is a lysine residue, wherein preferably said lysine residue is a lysine residue of a coat protein, preferably of a coat protein of an RNA-bacteriophage, most preferably of RNA-bacteriophage Qβ. In a further preferred embodiment said first attachment site is a lysine residue of the coat protein of RNA-bacteriophage Qβ.

Attachment Site, Second: As used herein, the phrase “second attachment site” refers to an element which is naturally occurring with or which is artificially added to the NGF antigen and to which the first attachment site may be linked. The second attachment site of the NGF antigen may be a protein, a polypeptide, a peptide, an amino acid, a sugar, a polynucleotide, a natural or synthetic polymer, a secondary metabolite or compound (biotin, fluorescein, retinol, digoxigenin, metal ions, phenylmethylsulfonylfluoride), or a chemically reactive group such as an amino group, a carboxyl group, a sulfhydryl group, a hydroxyl group, a guanidinyl group, histidinyl group, or a combination thereof A preferred embodiment of a chemically reactive group being the second attachment site is a sulfhydryl group. In a further preferred embodiment said second attachment site is a sulfhydryl group, preferably a sulfhydrly group of a cysteine residue. The terms “antigen with at least one second attachment site” and the interchangeably used term “NGF antigen with at least one second attachment site”, as used herein, refers to a construct comprising the NGF antigen and at least one second attachment site. In one preferred embodiment, the second attachment site is naturally occurring within the NGF antigen. In another preferred embodiment, the second attachment site is artificially added to the NGF antigen. In one preferred embodiment the second attachment site is associated with the NGF antigen through at least one covalent bond, preferably through at least one peptide bond. In one preferred embodiment, the NGF antigen with at least one second attachment site further comprises a linker, wherein preferably said linker comprises at least one second attachment site, wherein further preferably said linker is fused to the NGF antigen by a peptide bond.

Coat protein: The term “coat protein” refers to a viral protein, preferably a subunit of a natural capsid of a virus, preferably of an RNA-bacteriophage, which is capable of being incorporated into a virus capsid or a VLP. Coat proteins are also known as capsid proteins.

Linked: The term “linked” (or its noun: linkage) as used herein, refers to all possible ways, preferably chemical interactions, by which the at least one first attachment site and the at least one second attachment site are joined together. Chemical interactions include covalent and non-covalent interactions. Typical examples for non-covalent interactions are ionic interactions, hydrophobic interactions or hydrogen bonds, whereas covalent interactions are based, by way of example, on covalent bonds such as ester, ether, phosphoester, amide, peptide, carbon-phosphorus bonds, carbon-sulfur bonds such as thioether, or imide bonds. In certain preferred embodiments the first attachment site and the second attachment site are linked through at least one covalent bond, preferably through at least one non-peptide bond, and even more preferably exclusively through non-peptide bond(s). The term “linked” as used herein, however, shall not only encompass a direct linkage of the at least one first attachment site and the at least one second attachment site but also, alternatively and preferably, an indirect linkage of the at least one first attachment site and the at least one second attachment site through intermediate molecule(s), and hereby typically and preferably by using at least one, preferably one, heterobifunctional cross-linker Thus, in a preferred embodiment said least one first attachment site and said at least one second attachment site are covalently linked via least one, preferably exactly one, heterobifunctional cross-linker, wherein preferably said first attachment site is the amino group of a lysine residue and wherein further preferably said second attachment site is the sulfhydryl group of a cysteine residue.

Linker: A “linker”, as used herein, either associates the second attachment site with an NGF antigen or comprises, essentially consists of, or consists of the second attachment site. Preferably, a “linker”, as used herein, comprises the second attachment site, typically and preferably—but not necessarily—as one amino acid residue, preferably as a cysteine residue. In a preferred embodiment said linker is an amino acid linker. In a very preferred embodiment said linker consists of exactly one cysteine residue. In a further preferred embodiment said linker comprises or consists of exactly one cysteine residue and said second attachment site is the sulfhydryl group of said exactly one cysteine residue. Further linkers useful for the present invention are molecules comprising a C1-C6 alkyl-, a cycloalkyl such as a cyclopentyl or cyclohexyl, a cycloalkenyl, aryl or heteroaryl moiety. Moreover, linkers comprising preferably a C1-C6 alkyl-, cycloalkyl-(C5, C6), aryl- or heteroaryl-moiety and additional amino acid(s) can also be used as linkers for the present invention and shall be encompassed within the scope of the invention. Association of the linker with the NGF antigen is preferably by way of at least one covalent bond, more preferably by way of at least one peptide bond. In case of a second attachment site not naturally occurring with the NGF antigen, the linker is associated to the at least one second attachment site, for example, a cysteine, preferably, by way of at least one covalent bond, more preferably by way of at least one peptide bond.

Amino acid linker The term “amino acid linker” refers to a linker comprising at least one amino acid residue. Generally, the term “amino acid linker” does not imply that said amino acid linker would consists exclusively of amino acid residues. However, in a preferred embodiment said amino acid linker exclusively consists of amino acid residues. The amino acid residues of the linker are, preferably, composed of naturally occurring amino acids or non-natural amino acids known in the art, all-L or all-D, or mixtures thereof most preferably all-L. Further preferred embodiments of a linker in accordance with this invention are molecules comprising a sulfhydryl group or a cysteine residue and such molecules are, therefore, also encompassed within this invention.

NGF antigen: the term “NGF antigen” as used herein, refers to an NGF protein, an NGF fragment, or an NGF mutein. Very preferably, said NGF antigen is an NGF mutein. The term “NGF antigen”, as used herein, further encompasses post-translational modifications including but not limited to glycosylations, acetylations, phosphorylations of the NGF antigen as defined above. Typically and preferably, but not necessarily, said NGF antigen is specifically bound by monoclonal and/or polyclonal anti NGFβ antibodies.

NGF protein: The term “NGF protein” as used herein should encompass any polypeptide comprising, or alternatively or preferably consisting of, the human NGF of SEQ ID NO:22, the mouse NGF of SEQ ID NO:24, or the corresponding orthologs from any other animal. Very preferred NGF orthologs from various animal species are the polypeptides of SEQ ID NOs 32 to 39. An NGF protein typically, but not necessarily, comprises biological activity, preferably in a cell proliferation assay. Moreover, the term “NGF protein” as used herein should also encompass any polypeptide comprising, or alternatively or preferably consisting of, any natural or genetically engineered variant having more than 70%, preferably more than 80%, preferably more than 85%, even more preferably more than 90%, again more preferably more than 95%, and most preferably more than 97% amino acid sequence identity with the human NGF of SEQ ID NO:22, the mouse NGF of SEQ ID NO:24, or the corresponding orthologs from any other animal. The term “NGF protein” as used herein should furthermore encompass post-translational modifications including but not limited to glycosylations, acetylations, phosphorylations of the NGF protein as defined above. Preferably the NGF protein, as defined herein, consists of at most 200 amino acids in length, and even more preferably of at most 150 amino acids in length, still preferably at most 130 amino acids in length. Typically and preferably, said NGF protein is specifically bound by monoclonal and/or polyclonal anti NGFβ antibodies. Furthermore, NGF protein which is useful for the purpose of the invention is typically and preferably capable of inducing the formation of anti NGF antibodies in an animal, when administered to said animal in form of an immunogenic conjugate, preferably in form of a conjugate with a VLP of bacteriophage Qβ. Most preferred are NGF proteins which are capable of inducing the formation of anti NGF antibodies in an animal, when administered to said animal in form of an immunogenic conjugate, preferably in form of a conjugate with a VLP of bacteriophage Qβ, wherein said anti NGF antibodies are capable of neutralizing the biological activity of NGF protein in an in vitro and/or in an in vivo assay, preferably as described herein (cf. Examples 9 to 12 and 15). It is apparent for the artisan, that typically and preferably antibodies which are induced by a composition of the invention comprising an NGF protein of a certain species will be capable of specifically binding and/or neutralizing the NGF protein of said species.

NGF fragment: The term “NGF fragment”, as used herein, encompasses any polypeptide comprising, consisting essentially of, or alternatively or preferably consisting of, at least 8, preferably at least 12, more preferably at least 20, still more preferably at least 30 contiguous amino acids of an NGF protein and the same polypeptide comprising, consisting essentially of, or alternatively or preferably consisting of at most 60, more preferably at most 50, still more preferably at most 45, still more preferably at most 40 amino acids contiguous amino acids of a NGF protein as defined herein, as well as any polypeptide having more than 70%, more preferably more than 80%, still more preferably more than 90%, and even more preferably more than 95% amino acid sequence identity thereto. In a preferred embodiment said NGF fragment comprises, consists essentially of, or preferably consists of a polypeptide selected from (a) a polypeptide consisting of 8 to 60, preferably of 12 to 60, more preferably of 20 to 60, still more preferably of 30 to 60 contiguous amino acids of an NGF protein as defined herein, preferably of SEQ ID NO:22, and (b) a polypeptide having more than 70%, preferably more than 80%, more preferably more than 90%, and even more preferably more than 95% amino acid sequence identity to the polypeptide of (a). Preferably, the term “NGF fragment” as used herein encompasses any polypeptide comprising, consisting essentially of, or alternatively or preferably consisting of, at least 12 contiguous amino acids of the NGF protein of SEQ ID NO:22 and the same polypeptide comprising, consisting essentially of, or alternatively or preferably consisting of at most 45, still more preferably at most 40 contiguous amino acids of the NGF protein of SEQ ID NO:22. The term “NGF fragment” as used herein should furthermore encompass post-translational modifications including but not limited to glycosylations, acetylations, phosphorylations of the NGF fragment as defined above. An NGF fragment typically, but not necessarily, comprises biological activity, preferably in a cell proliferation assay. Typically and preferably, said NGF fragment is specifically bound by monoclonal and/or polyclonal anti NFGβ antibodies. Furthermore, NGF fragments which are useful for the purpose of the invention are typically and preferably capable of inducing the formation of anti NGF antibodies in an animal, when administered to said animal in form of an immunogenic conjugate, preferably in form of a conjugate with a VLP of bacteriophage Qβ. Most preferred are NGF fragments which are capable of inducing the formation of anti NGF antibodies in an animal, when administered to said animal in form of an immunogenic conjugate, preferably in form of a conjugate with a VLP of bacteriophage Qβ, wherein said anti NGF antibodies are capable of neutralizing the biological activity of NGF protein in an in vitro and/or in an in vivo assay, preferably as described herein (cf. Examples 9 to 12 and 15). It is apparent for the artisan, that typically and preferably antibodies which are induced by a composition of the invention comprising an NGF fragment of a certain species will be capable of specifically binding and/or neutralizing the NGF protein of said species.

NGF mutein: The term “NGF mutein”, as used herein encompasses any polypeptide comprising, or alternatively or preferably consisting of, a mutated amino acid sequence, wherein the amino acid sequence to be mutated is an NGF protein, preferably the human NGF protein, most preferably SEQ ID NO:22, and wherein said amino acid sequence to be mutated is altered in at least one and in at most 20, preferably in at least one and in at most 10, more preferably in at least one and in at most 5 positions, wherein the amino acid residues in said positions are altered by substitution, by deletion, by insertion, or by any combination thereof. In a very preferred embodiment, said NGF mutein is a mutated amino acid sequence, wherein the amino acid sequence to be mutated is SEQ ID NO:22, and wherein said amino acid sequence to be mutated is altered in at least one and in at most 4 positions by substitution, by deletion or by any combination thereof, wherein preferably the amino acid residues in said positions are altered by substitution. Typically, but not necessarily, said NGF mutein is specifically bound by monoclonal and/or polyclonal anti NGFβ antibodies. An NGF mutein comprises reduced biological activity as compared to said amino acid sequence to be mutated. Most preferably, said NGF mutein does not comprise detectable biological activity. Furthermore, an NGF mutein which is useful for the purpose of the invention is typically and preferably capable of inducing the formation of anti NGF antibodies in an animal, when administered to said animal in form of an immunogenic conjugate, preferably in form of a conjugate with a VLP of bacteriophage Qβ. Most preferred are NGF muteins which are capable of inducing the formation of anti NGF antibodies in an animal, when administered to said animal in form of an immunogenic conjugate, preferably in form of a conjugate with a VLP of bacteriophage Qβ, wherein said anti NGF antibodies are capable of neutralizing the biological activity of NGF protein in an in vitro and/or in an in vivo assay, preferably as described herein (cf. Examples 9 to 12). It is apparent for the artisan, that typically and preferably antibodies which are induced by a composition of the invention comprising an NGF mutein, wherein said amino acid sequence to be mutated is of a certain species will be capable of specifically binding and/or neutralizing the NGF protein of said species.

Within this application specific binding of an antibody, preferably of a monoclonal or a polyclonal antibody, to the antigen refers to a binding which is characterized by an affinity (Ka) of 10⁶ M⁻¹ or greater, preferably 10⁷ M⁻¹ or greater, more preferably 10⁸ M⁻¹ or greate and most preferably 10⁹ M⁻¹ or greater. The affinity of an antibody can be readily determined by one of ordinary skill in the art (for example, by Scatchard analysis, Biacore- or ELISA-based methods). Most preferably, the specific binding of a monoclonal and/or polyclonal anti NGF antibody to an NGF antigen is assayed by ELISA, most preferably under conditions essentially as described in Example 5 herein.

Biological activity: The term “biological activity” as used herein refers to the activity of an antigen, preferably of an NGF antigen, most preferably of an NGF protein, an NGF fragment and/or an NGF mutein in a cell proliferation assay, wherein preferably said cell proliferation assay is based on an NGF dependent human erythroleukemic TF-1 cell line, wherein still further preferably said cell proliferation assay is performed under conditions essentially as described in Example 6 herein. An NGF antigen is regarded as being biologically active, if it is capable of inducing a detectable level of cell proliferation. Typically and preferably, an NGF antigen is biologically active if it is capable of inducing cell proliferation which is at least 20%, preferably at least 50, still more preferably at least 80% of the maximal proliferation, which is achieved with an appropriate standard. An NGF antigen, preferably an NGF mutein, is not biologically active if it is not inducing a detectable level of cell proliferation. Typically and preferably, an NGF antigen is not biologically active if it is inducing cell proliferation which is at most 20%, preferably at most 15%, more preferably at most 10%, still more preferably at most 5% of the maximal proliferation, which is achieved with an appropriate standard. An NGF mutein is regarded as comprising reduced biologically activity if it induces cell proliferation which is less than 100%, preferably less than 80%, still more preferably less than 60%, still more preferably less than 40%, still more preferably less than 20% as compared to a polypeptide consisting said amino acid sequence to be mutated.

In the context of this invention an NGF antigen and in particular an NGF protein, an NGF fragment and an NGF mutein is regarded as being “capable of inducing antibodies”, if it induces an antibody response, when administered to a test animal in form of an immunogenic conjugate, wherein the antibodies produced in said antibody response are specifically binding said NGF antigen. Typically and preferably said NGF protein is administered to said test animal in form of a conjugate with a virus-like particle, most preferably in form of a conjugate with a virus-like particle of RNA-bacteriophage Qβ. Most preferably, said NGF antigen is administered to said test animal in form of a composition, a vaccine composition or a pharmaceutical composition as disclosed herein. Very preferably, the capability of an NGF antigen to induce antibodies is assayed under conditions essentially as set forth in Example 8.

In the context of this invention an NGF antigen and in particular an NGF protein, an NGF fragment and an NGF mutein is regarded as being “capable of inducing neutralizing antibodies”, if it induces an antibody response, when administered to a test animal in form of an immunogenic conjugate, wherein the antibodies produced in said antibody response are capable of neutralizing NGF protein. The capability of an antibody to neutralize an NGF protein is assayed in an in vitro assay using a cell proliferation assay, wherein preferably said cell proliferation assay is based on an NGF dependent human erythroleukemic TF-1 cell line, wherein still further preferably said cell proliferation assay is performed under conditions essentially as described in Examples 6 and 9 herein. An antibody is regarded as capable of neutralizing an NGF protein when it reduces or eliminates the biological activity of said NGF protein in said cell proliferation assay. Alternatively, the capability of an antibody to neutralize an NGF protein is assayed in an in vivo assay, preferably in an animal model for pain, wherein the capability of said antibody to neutralize an NGF protein is ultimately detected as the amelioration of pain in said animal model. Preferred animal models for pain are collagen induced arthritis in mice, zymosan A-induced inflammatory pain in mice, taxol-induced neuropathic pain, wherein preferably said assay is performed essentially under conditions as disclosed in Examples 10, 11, or 15.

Ordered and repetitive antigen array: As used herein, the term “ordered and repetitive antigen array” generally refers to a repeating pattern of antigen or, characterized by a typically and preferably high order of uniformity in spacial arrangement of the antigens with respect to virus-like particle, respectively. In one embodiment of the invention, the repeating pattern may be a geometric pattern. Certain embodiments of the invention, such as VLP of RNA-bacteriophages, are typical and preferred examples of suitable ordered and repetitive antigen arrays which, moreover, possess strictly repetitive paracrystalline orders of antigens, preferably with spacings of 1 to 30 nanometers, preferably 2 to 15 nanometers, even more preferably 2 to 10 nanometers, even again more preferably 2 to 8 nanometers, and further more preferably 1.6 to 7 nanometers.

Polypeptide: The term “polypeptide” as used herein refers to a molecule composed of monomers (amino acids) linearly linked by amide bonds (also known as peptide bonds). It indicates a molecular chain of amino acids and does not refer to a specific length of the product. Thus, peptides, dipeptides, tripeptides, oligopeptides and proteins are included within the definition of polypeptide. Post-translational modifications of the polypeptide, for example, glycosylations, acetylations, phosphorylations, and the like are also encompassed.

The amino acid sequence identity of polypeptides can be determined conventionally using known computer programs such as the Bestfit program. When using Bestfit or any other sequence alignment program, preferably using Bestfit, to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, polypeptides or fragments thereof disclosed in this invention.

Conservative amino acid substitutions, as understood by the artisan, include isosteric substitutions, i.e. substitutions wherein the charged, polar, aromatic, aliphatic or hydrophobic nature of the amino acid is maintained. Typical conservative amino acid substitutions are substitutions between amino acids within one of the following groups: (a) Gly, Ala; Val, Ile, and Leu; (b) Asp, Glu; Asn, Gln; Ser, Thr, Cys; Lys, and Arg; and (c) Phe and Tyr.

Virus particle: The term “virus particle” as used herein refers to the morphological form of a virus. In some virus types it comprises a genome surrounded by a protein capsid; others have additional structures (e.g., envelopes, tails, etc.).

Virus-like particle (VLP): The term virus-like particle as used herein, refers to a non-replicative or non-infectious, preferably a non-replicative and non-infectious virus particle, or refers to a non-replicative or non-infectious, preferably a non-replicative and non-infectious structure resembling a virus particle, preferably a capsid of a virus. The term “non-replicative”, as used herein, refers to being incapable of replicating the genome comprised by the VLP. The term “non-infectious”, as used herein, refers to being incapable of entering the host cell. Preferably a virus-like particle in accordance with the invention is non-replicative and/or non-infectious since it lacks all or part of the viral genome or genome function due to physical, chemical inactivation or due to genetic manipulation. Typically and preferably a virus-like particle lacks all or part of the replicative and infectious components of the viral genome. A virus-like particle in accordance with the invention may contain nucleic acids distinct from the viral genome. A typical and preferred embodiment of a virus-like particle in accordance with the present invention is a viral capsid such as the viral capsid of the corresponding virus, bacteriophage, preferably RNA-bacteriophage. The terms “viral capsid” or “capsid”, refer to a macromolecular assembly composed of viral protein subunits. Typically, there are 60, 120, 180, 240, 300, 360 and more than 360 viral protein subunits. Typically and preferably, the interactions of these subunits lead to the formation of viral capsid or viral-capsid like structure with an inherent repetitive organization, wherein said structure is, typically, spherical or tubular.

Recombinant VLP: The term “recombinant VLP”, as used herein, refers to a VLP that is obtained by a process which comprises at least one step of recombinant DNA technology.

Packaged: The term “packaged” as used herein refers to the state of a polyanionic macromolecule in relation to the VLP. The term “packaged” as used herein includes binding that may be covalent, e.g., by chemically coupling, or non-covalent, e.g., ionic interactions, hydrophobic interactions, hydrogen bonds, etc. In a preferred embodiment, the term “packaged” refers to the enclosement, or partial enclosement, of a polyanionic macromolecule by the VLP. Thus, the polyanionic macromolecule can be enclosed by the VLP without the existence of an actual binding, in particular of a covalent binding. In preferred embodiments, at least one polyanionic macromolecule is packaged into said VLP, most preferably in a non-covalent manner. Methods for packaging polyanionic macromolecules such as polyglutamic acid into VLPs, and in particular into VLPs of RNA-bacteriophages, are disclosed in WO2006/037787. Reference is made in particular to Example 4 of WO2006/037787.

Polyanionic macromolecule: The term “polyanionic macromolecule”, as used herein, refers to a molecule of high relative molecular mass which comprises repetitive groups of negative charge, the structure of which essentially comprises the multiple repetition of units derived, actually or conceptually, from molecules of low relative molecular mass. The term “polyanionic macromolecule” as used herein refers to a molecule that is not capable of activating toll-like receptors. Thus, the term “polyanionic macromolecule” excludes Toll-like receptors ligands, and excludes substances capable of inducing and/or enhancing an immune response, such as Toll-like receptors ligands, nucleic acids capable of inducing and/or enhancing an immune response, and lipopolysacchrides (LPS). More preferably the term “polyanionic macromolecule” as used herein, refers to a molecule that is not capable of inducing cytokine production. Preferably, polyanionic macromolecules are polyanionic polypeptides or anionic dextrans. In a preferred embodiment said polyanionic macromolecules are polyanionic polypeptides, wherein preferably said polyanionic polypeptides are selected from a group consisting of: (a) polyglutamic acid; (b) polyaspartic acid; (c) poly(GluAsp) and (d) any chemical modifications of (a) to (c). Examples for chemical modifications include, but are not limited to glycosylations, acetylations, and phosphorylations. In a further preferred embodiment said polyanionic macromolecules are anionic dextrans selected from a group consisting of: (a) dextran sulfate; (b) carboxylmethyl dextran; (c) sulfopropyl dextran;(d) methyl sulfonate dextran; and (e) dextrane phosphate.

Polyaspartic acid: The term “polyaspartic acid” as used herein, refers to a polypeptide comprising at least 50%, preferably at least 70%, more preferably at least 90%, more preferably at least 95%, more preferably at least 99%, more preferably 100%, aspartic acid residues out of the total number of amino acid residues comprised by said polypeptide. The aspartic acid residues of said polypeptide are hereby either all-L, all-D, or mixtures of L- and D-aspartic acid. Most preferably said polypeptide only comprises L-aspartic acid residues.

Polyglutamic acid: The term “polyglutamic acid”, as used herein, refers to a polypeptide comprising at least 50%, preferably at least 70%, more preferably at least 90%, more preferably at least 95%, more preferably at least 99%, and most preferably 100% glutamic acid residues out of the total number of amino acid residues comprised by said polypeptide. The glutamic acid residues of said polypeptide are hereby either all-L, all-D, or mixtures of L- and D-glutamic acid. Most preferably said polypeptide only comprises L-glutamic acid residues.

Poly (GluAsp): The term “Poly (GluAsp)” as used herein, refers to a polypeptide comprising at least 50%, preferably at least 70%, more preferably at least 90% , still more preferably at least 95%, still more preferably at least 99%, and most preferably 100% glutamic acid residues and aspartic acid residues, out of the total number of amino acid residues comprised by said polypeptide. The glutamic acid molecules and the aspartic acid molecules are hereby either all-L or all-D or mixtures thereof. Most preferably said polypeptide only comprises L-glutamic acid residues and L-aspartic acid residues.

Pain: pain either arises from the stimulation of pain receptors (nociceptive pain), e.g. by injury, or it is caused by a malfunction in the nervous system that leads to a pain signal being sent to the brain, without obvious physical cause (neuropathic pain). Pain includes, for example, ostheoarthritic pain, rheumatoid arthritis pain, cancer pain, visceral pain, chronic low back pain, and chronic headache, fibromyalgia, diabetic neuropathy, phantom limb pain and post herpetic neuralgia.

Nociceptive pain: The term “nociceptive pain” as used herein, refers to a pain arising from the stimulation of the pain receptors, due to injury, surgery or disease that affect the tissues, such as arthritis and cancer. Nociceptive pain also includes chronic pain. Preferred types of nociceptive pain are ostheoarthritic pain, rheumatoid arthritis pain, cancer pain, visceral pain, chronic low back pain, and chronic headache.

Neuropathic pain: The term “neuropathic pain” as used herein, refers to a pain caused by a malfunction in the nervous system that leads to a pain signal being sent to the brain, without obvious physical cause, such as diabetic neuropathy, phantom limb pain and post herpetic neuralgia.

One common feature of virus particles and virus-like particles is the highly ordered and repetitive arrangement of their subunits.

Virus-like particle of an RNA-bacteriophage: As used herein, the term “virus-like particle of an RNA-bacteriophage” refers to a virus-like particle comprising, or preferably consisting essentially of or consisting of coat proteins, mutants or fragments thereof, of an RNA-bacteriophage. In addition, a virus-like particle of an RNA-bacteriophage is resembling the structure of an RNA-bacteriophage. Furthermore, a virus-like particle of an RNA-bacteriophage is non-replicative and non-infectious. Typically and preferably, the term “virus-like particle of an RNA-bacteriophage” furthermore refers to a virus-like particle of an RNA-bacteriophage which lacks at least one of the genes, preferably all of the genes, encoding for the replication machinery of the RNA-bacteriophage, and typically and further preferably even at least one of the genes, preferably all of the genes, encoding the protein or proteins responsible for viral attachment to or entry into the host. This definition however also encompasses virus-like particles of RNA-bacteriophages, wherein the aforementioned gene or genes are still present but inactive, and, therefore, lead to non-replicative and noninfectious virus-like particles of an RNA-bacteriophage. In its broadest definition the term “virus-like particle of an RNA-bacteriophage” therefore also encompasses a virus particle of an RNA-bacteriophage, the genome of which has been inactivated by physical or chemical or genetic methods so that the virus particle is non-replicative and/or non-infectious. Preferred VLPs of RNA-bacteriophages exhibit icosahedral symmetry and consist of 180 subunits.

One, a, or an: when the terms “one”, “a”, or “an” are used in this disclosure, they mean “at least one” or “one or more” unless otherwise indicated.

This invention provides compositions and methods for enhancing immune responses against NGF in an animal or in human. Composition of the invention comprises: (a) a virus-like particle (VLP) with at least one first attachment site; and (b) at least one antigen with at least one second attachment site, wherein the at least one antigen is a NGF antigen and wherein (a) and (b) are linked through the at least one first and the at least one second attachment site. Preferably, the NGF antigen is linked to the VLP, so as to form an ordered and repetitive antigen-VLP array. In preferred embodiments of the invention, at least 20, preferably at least 30, more preferably at least 60, still more preferably at least 120, and most preferably at least 180 NGF antigens are linked to the VLP.

Any virus known in the art having an ordered and repetitive structure may be selected as a VLP of the invention. Illustrative DNA or RNA viruses, the coat protein of which can be used for the preparation of VLPs have been disclosed in WO 2004/009124 on page 25, line 10-21, on page 26, line 11-28, and on page 28, line 4 to page 31, line 4. These disclosures are incorporated herein by way of reference.

Viruses or virus-like particles can be produced and purified from virus-infected cell cultures. For the purpose of vaccination the resulting viruses or virus-like particles need to be devoid of virulence. Besides genetic engineering, physical or chemical methods can be employed to inactivate the viral genome function, such as UV irradiation, formaldehyde treatment.

In one preferred embodiment, the VLP is a recombinant VLP. Almost all commonly known viruses have been sequenced and are readily available to the public. The gene encoding the coat protein can be easily identified by a skilled artisan. The preparation of VLPs by recombinantly expressing the coat protein in a host is within the common knowledge of a skilled artisan.

In one preferred embodiment, the virus-like particle comprises, or alternatively consists of, recombinant proteins, mutants or fragments thereof, of a virus selected form the group consisting of: (a) RNA-bacteriophages; (b) bacteriophages; (c) Hepatitis B virus, preferably its coat protein (Ulrich, et al., Virus Res. 50:141-182 (1998)) or its surface protein (WO 92/11291); d) measles virus (Warnes, et al., Gene 160:173-178 (1995)); (e) Sindbis virus; (f) rotavirus (U.S. Pat. No. 5,071,651 and U.S. Pat. No. 5,374,426); (g) foot-and-mouth-disease virus (Twomey, et al., Vaccine 13:1603 1610, (1995)); (h) Norwalk virus (Jiang, X., et al., Science 250:1580 1583 (1990); Matsui, S. M., et al., J. Clin. Invest. 87:1456 1461 (1991)); (i) Alphavirus; j) retrovirus, preferably its GAG protein (WO 96/30523); (k) retrotransposon Ty, preferably the protein pl; (1) human Papilloma virus (WO 98/15631); (m) Polyoma virus; (n) Tobacco mosaic virus; and (o) Flock House Virus.

In one preferred embodiment, the VLP comprises or consists of more than one amino acid sequence, preferably of two amino acid sequences, of the recombinant proteins, mutants or fragments thereof In a further preferred embodiment, the VLP comprises or consists of at least one first polypeptide and of at least one second polypeptide, wherein said first and said second polypeptide comprise an amino acid sequence of a coat protein, or of mutants or fragments thereof, wherein the amino acid sequence of said first polypeptide and of said second polypeptide are not identical. In a further preferred embodiment, said first polypeptide comprise an amino acid sequence of a first coat protein, or of mutants or fragments thereof, and said second polypeptide comprise an amino acid sequence of a second coat protein, or of mutants or fragments thereof, wherein said first coat protein and said second coat protein are coat proteins of the same virus, and wherein preferably said virus is an RNA-bacteriophage. A VLP which comprises or consists of more than one polypeptide species is referred to as mosaic VLP.

The term “fragment of a recombinant protein” or the term “fragment of a coat protein”, as used herein, is defined as a polypeptide, which is of at least 70%, preferably at least 80%, more preferably at least 90%, even more preferably at least 95% the length of the wild-type recombinant protein, or coat protein, respectively and which preferably retains the capability of forming VLP. Preferably, the fragment is obtained by at least one internal deletion, at least one truncation or at least one combination thereof. Further preferably, the fragment is obtained by at most 5, 4, 3 or 2 internal deletions, by at most 2 truncations or by exactly one combination thereof.

The term “fragment of a recombinant protein” or “fragment of a coat protein” shall further encompass a polypeptide which has at least 80%, preferably at least 90%, most preferably at least 95% amino acid sequence identity with the “fragment of a recombinant protein” or “fragment of a coat protein”, respectively, as defined above, and which is preferably capable of assembling into a virus-like particle.

The term “mutant recombinant protein” or the term “mutant of a recombinant protein” as interchangeably used in this invention, or the term “mutant coat protein” or the term “mutant of a coat protein”, as interchangeably used in this invention, refers to a polypeptide having an amino acid sequence derived from the wild type recombinant protein, or coat protein, respectively, wherein the amino acid sequence is at least 80%, preferably at least 85%, at least 90%, at least 95%, at least 97%, or at least 99% identical to the wild type sequence, and wherein preferably said polypeptide retains the ability to assemble into a VLP.

In one preferred embodiment, the virus-like particle of the invention is of Hepatitis B virus. The preparation of Hepatitis B virus-like particles is disclosed, inter alia, in WO 00/32227, WO 01/85208 and in WO 01/056905. All three documents are explicitly incorporated herein by way of reference. Other variants of HBcAg suitable for use in the practice of the present invention are disclosed in WO 01/056905, in particular on pages 34 to 39 therein.

In a further preferred embodiment of the invention, a lysine residue is introduced into the HBcAg polypeptide, to mediate the linking of NGF antigen to the VLP of HBcAg. In preferred embodiments, VLPs and compositions of the invention are prepared using a HBcAg comprising, or alternatively consisting of, amino acids 1-144, or 1-149, 1-185 of SEQ ID NO:20, which is modified so that the amino acids at positions 79 and 80 are replaced with a peptide having the amino acid sequence of Gly-Gly-Lys-Gly-Gly. This modification changes the SEQ ID NO:20 to SEQ ID NO:21. In further preferred embodiments, the cysteine residues at positions 48 and 110 of SEQ ID NO:21, or its corresponding fragments, preferably 1-144 or 1-149, are mutated to serine. The invention further includes compositions comprising Hepatitis B core protein mutants having above noted corresponding amino acid alterations. The invention further includes compositions and vaccines, respectively, comprising HBcAg polypeptides which comprise, or alternatively consist of, amino acid sequences which are at least 80%, 85%, 90%, 95%, 97% or 99% identical to SEQ ID NO:21.

In one preferred embodiment of the invention, the virus-like particle is a virus-like particle of an RNA-bacteriophage, wherein preferably said RNA-bacteriophage is selected from the group consisting of (a) bacteriophage Qβ; (b) bacteriophage R17; (c) bacteriophage fr; (d) bacteriophage GA; (e) bacteriophage SP; (f) bacteriophage MS2; (g) bacteriophage M11; (h) bacteriophage MX1; (i) bacteriophage NL95; (k) bacteriophage f2; (1) bacteriophage PP7, and (m) bacteriophage AP205. In a very preferred embodiment said virus-like particle is a virus-like particle of RNA-bacteriophage Qβ.

In a further preferred embodiment, said virus-like particle comprises, consists essentially of, or alternatively consists of, recombinant coat proteins, mutants or fragments thereof, of an RNA-bacteriophage, wherein preferably said RNA-bacteriophage is selected from the group consisting of (a) bacteriophage Qβ; (b) bacteriophage R17; (c) bacteriophage fr; (d) bacteriophage GA; (e) bacteriophage SP; (f) bacteriophage MS2; (g) bacteriophage M11; (h) bacteriophage MX1; (i) bacteriophage NL95; (k) bacteriophage f2; (1) bacteriophage PP7, and (m) bacteriophage AP205. In a further preferred embodiment said virus-like particle comprises, or alternatively consists essentially of, or alternatively consists of recombinant coat proteins, mutants or fragments thereof, of an RNA-bacteriophage, wherein said RNA bacteriophage is selected from Qβ, fr, AP205 or GA. In a very preferred embodiment said virus-like particle comprises, consists essentially of, or alternatively consists of recombinant coat proteins, mutants or fragments thereof, of RNA-bacteriophage Qβ.

In one preferred embodiment of the invention, the composition comprises coat proteins, mutants or fragments thereof, of RNA-bacteriophages, wherein preferably said coat proteins comprise or preferably consist of an amino acid sequence selected from the group consisting of: (a) SEQ ID NO:1; referring to Qβ CP; (b) a mixture of SEQ ID NO:1 and SEQ ID NO:2 (referring to Qβ A1 protein); (c) SEQ ID NO:3; (d) SEQ ID NO:4; (e) SEQ ID NO:5; (f) SEQ ID NO:6, (g) a mixture of SEQ ID NO:6 and SEQ ID NO:7; (h) SEQ ID NO:8; (i) SEQ ID NO:9; (j) SEQ ID NO:10; (k) SEQ ID NO:1 l; (1) SEQ ID NO:12; (m) SEQ ID NO:13; (n) SEQ ID NO:14; (o) SEQ ID NO:40; (p) SEQ ID NO:41; and (q) SEQ ID NO:42.

In a further preferred embodiment, said virus-like particle comprises, consists essentially of, or alternatively consists of recombinant coat proteins, mutants or fragments thereof, of RNA-bacteriophages. In a further preferred embodiment, said virus-like particle comprises, consists essentially of, or alternatively consists of recombinant coat proteins, mutants or fragments thereof, of RNA-bacteriophages, wherein said coat proteins comprise or preferably consist of an amino acid sequence selected from the group consisting of: (a) SEQ ID NO:1; referring to Qβ CP; (b) a mixture of SEQ ID NO:1 and SEQ ID NO:2 (referring to Qβ A1 protein); (c) SEQ ID NO:3; (d) SEQ ID NO:4; (e) SEQ ID NO:5; (f) SEQ ID NO:6, (g) a mixture of SEQ ID NO:6 and SEQ ID NO:7; (h) SEQ ID NO:8; (i) SEQ ID NO:9; (j) SEQ ID NO:10; (k) SEQ ID NO:11; (1) SEQ ID NO:12; (m) SEQ ID NO:13; and (n) SEQ ID NO:14; (o) SEQ ID NO:40; (p) SEQ ID NO:41; and (q) SEQ ID NO:42.

In a further preferred embodiment, said virus-like particle comprises, consists essentially of, or alternatively consists of recombinant coat proteins, wherein said recombinant coat proteins comprise or preferably consist of the amino acid sequence of SEQ ID NO:1.

In one preferred embodiment of the invention, the VLP is a mosaic VLP comprising or alternatively consisting of more than one amino acid sequence, preferably of two amino acid sequences, of coat proteins, mutants or fragments thereof, of an RNA-bacteriophage. In one very preferred embodiment, the VLP comprises or alternatively consists of two different coat proteins of an RNA-bacteriophage, wherein said two different coat proteins comprise or preferably consist of the amino acid sequences of SEQ ID NO:1 and SEQ ID NO:2, or of the amino acid sequences of SEQ ID NO:6 and SEQ ID NO:7, respectively.

In one preferred embodiment said VLP is a VLP of RNA-bacteriophage Qβ. The capsid or virus-like particle of Qβ show an icosahedral phage-like capsid structure with a diameter of 25 nm and T=3 quasi symmetry. The capsid contains 180 copies of the coat protein, which are linked in covalent pentamers and hexamers by disulfide bridges (Golmohammadi, R. et al., Structure 4:543-5554 (1996)), leading to a remarkable stability of the Qβ capsid. Capsids or VLPs made from recombinant Qβ coat protein may contain, however, subunits not linked via disulfide bonds to other subunits within the capsid, or incompletely linked. The capsid or VLP of Qβ shows unusual resistance to organic solvents and denaturing agents. Surprisingly, we have observed that DMSO and acetonitrile concentrations as high as 30%, and guanidinium concentrations as high as 1 M do not affect the stability of the capsid. The high stability of the capsid or VLP of Qβ is an advantageous feature, in particular, for its use in immunization and vaccination of mammals and humans in accordance of the present invention.

Further preferred virus-like particles of RNA-bacteriophages, in particular of RNA-bacteriophage Qβ and RNA-bacteriophage fr, in accordance with this invention are disclosed in WO 02/056905, the disclosure of which is herewith incorporated by reference in its entirety. In particular, Example 18 of WO 02/056905 provides a detailed description of preparation of VLPs of RNA-bacteriophage Qβ.

In another preferred embodiment, said VLP is a VLP of RNA-bacteriophage AP205. Assembly-competent mutant forms of AP205 VLPs, including AP205 coat protein with the substitution of proline at amino acid 5 to threonine or AP205 coat protein with the substitution of asparagine at amino acid 14 to aspartic acid, may also be used in the practice of the invention and leads to other preferred embodiments of the invention. WO 2004/007538 describes, in particular in Example 1 and Example 2, how to obtain VLPs comprising AP205 coat proteins, and hereby in particular the expression and the purification thereof. WO 2004/007538 is incorporated herein by way of reference. AP205 VLPs are highly immunogenic, and can be linked with antigen to typically and preferably generate vaccine constructs displaying the antigen in an oriented and repetitive manner. High antibody titer is elicited against the so displayed antigens showing that linked antigens are capable of interacting with cells of the immune system, typically and preferably with B-cells, and thus, are immunogenic.

In one preferred embodiment, said VLP comprises or consists of a mutant coat protein of a virus, preferably of an RNA-bacteriophage, wherein the mutant coat protein has been modified by removal of at least one lysine residue by way of substitution and/or by way of deletion. In another preferred embodiment, the VLP of the invention comprises or consists of a mutant coat protein of a virus, preferably of an RNA-bacteriophage, wherein the mutant coat protein has been modified by addition of at least one lysine residue by way of substitution and/or by way of insertion. The deletion, substitution or addition of at least one lysine residue allows varying the degree of coupling, i.e. it allows varying the amount of antigen which is bound per subunits of the VLP, preferably of the VLP of an RNA-bacteriophage. Thus, the requirements for the design of a specific vaccine can be met.

In one preferred embodiment, the compositions and vaccines of the invention have an antigen density of 0.5 to 4.0. The term “antigen density”, as used herein, refers to the average number of antigen molecules which is linked per subunit, preferably per coat protein, of the VLP, and hereby preferably of the VLP of an RNA-bacteriophage. Thus, this value is calculated as an average over all subunits of said VLP, preferably of said VLP of an RNA-bacteriophage, in the composition or vaccines of the invention.

VLPs or capsids of Qβ coat protein display a defined number of lysine residues on their surface, with a defined topology with three lysine residues pointing towards the interior of the capsid and interacting with the RNA, and four other lysine residues exposed to the exterior of the capsid. Preferably, the at least one first attachment site is a lysine residue, pointing to or being on the exterior of the VLP. More preferably, the at least one first attachment site is the amino group of a lysine residue pointing to or being on the exterior of the VLP. In a further preferred embodiment said first attachment site is an amino group of a lysine residue of the coat protein of RNA-bacteriophage Qβ. In a further preferred embodiment said first attachment site is an amino group of a lysine residue of SEQ ID NO:1. In a further preferred embodiment said first attachment site is the amino group of any one of the lysine residues in positions 2, 13, 16, 46, 60, 63, and 67 of SEQ ID NO:1. In a further preferred embodiment said first attachment site is the amino group of any one of the lysine residues exposed to the exterior of the capsid.

Qβ mutants wherein exposed lysine residues are replaced by arginines can be used for the present invention. Thus, in another preferred embodiment of the present invention, the virus-like particle comprises, consists essentially of or alternatively consists of mutant Qβ coat proteins. Preferably, these mutant coat proteins comprise or alternatively consist of an amino acid sequence selected from the group consisting of (a) Qβ-240 (SEQ ID NO:15, Lys13-Arg of SEQ ID NO:1); (b) Qβ-243 (SEQ ID NO:16, Asn10-Lys of SEQ ID NO:1); (c) Qβ-250 (SEQ ID NO:17, Lys2-Arg of SEQ ID NO:1); (d) Qβ-251 (SEQ ID NO:18, Lys16-Arg of SEQ ID NO:1); and (e) Qβ-259 (SEQ ID NO:19, Lys2-Arg, Lys16-Arg of SEQ ID NO:1). The construction, expression and purification of the above indicated Qβ mutant coat proteins, mutant Qβ coat protein VLPs and capsids, respectively, are described in WO 02/056905. In particular it is hereby referred to Example 18 of a WO 02/056905.

In another preferred embodiment of the present invention, the virus-like particle comprises, or alternatively consists essentially of, or alternatively consists of mutant coat protein of Qβ, or mutants or fragments thereof, and of the corresponding A1 protein. In a further preferred embodiment, the virus-like particle comprises, or alternatively consists essentially of, or alternatively consists of mutant coat protein, wherein said mutant coat protein is selected from any one of SEQ ID NOs 15, 16, 17, 18, and 19, and of the corresponding A1 protein.

Further RNA-bacteriophage coat proteins have also been shown to self-assemble upon expression in a bacterial host (Kastelein, R A. et al., Gene 23:245-254 (1983), Kozlovskaya, T M. et al., Dokl. Akad. Nauk SSSR 287:452-455 (1986), Adhin, M R. et al., Virology 170:238-242 (1989), Priano, C. et al., J. Mol. Biol. 249:283-297 (1995)). In particular, the biological and biochemical properties of GA (Ni, C Z., et al., Protein Sci. 5:2485-2493 (1996), Tars, K et al., J. Mol. Biol. 271:759-773(1997)) and of fr (Pushko P. et al., Prot. Eng. 6:883-891 (1993), Liljas, L et al. J Mol. Biol. 244:279-290, (1994)) have been disclosed. The crystal structure of several RNA-bacteriophages has been determined (Golmohammadi, R. et al., Structure 4:543-554 (1996)). Using such information, surface exposed residues can be identified and, thus, coat proteins of RNA-bacteriophages can be modified such that one or more reactive amino acid residues can be inserted by way of insertion or substitution. Another advantage of the VLPs derived from RNA-bacteriophages is their high expression yield in bacteria that allows production of large quantities of material at affordable cost.

In one preferred embodiment, the NGF antigen is a NGF protein. In one preferred embodiment, the NGF protein is selected from the group consisting of: (a) a human NGF protein; (b) a dog NGF protein; (c) a feline NGF protein; (d) a mouse NGF protein, and (e) a horse NGF protein. In one further preferred embodiment, the NGF protein is the NGF of human or of other animals, preferably of mammals, wherein further preferably said NGF protein is selected from any one of SEQ ID NOs 22 to 25 and 32 to 39.

In one preferred embodiment, the NGF protein is the human NGF. In one further preferred embodiment, the human NGF comprises or preferably consists of the amino acid sequence set forth in SEQ ID NO:22. In another preferred embodiment, the NGF protein comprises or preferably consists of the an amino acid sequence, which is at least 80%, or preferably at least 85%, more preferably at least 90%, or most preferably at least 95% identical with SEQ ID NO:22.

In one preferred embodiment, the NGF protein comprises or preferably consists of an amino acid sequence, in which SEQ ID NO:22 is altered by at least one and by at most 20, preferably by at least one and by at most 10, more preferably by at least one and by at most 5 amino acids by substitution and/or deletion and/or insertion or a combination thereof.

In a further preferred embodiment, the NGF protein comprises or preferably consists of a mutated amino acid sequence, wherein the amino acid sequence to be mutated is SEQ ID NO:22, and wherein said amino acid sequence to be mutated is altered in at least one and in at most 20, preferably in at least one and in at most 10, more preferably in at least one and in at most 5 positions, wherein the amino acid residues in said positions are altered by substitution, by deletion, by insertion, or by any combination thereof.

In one preferred embodiment, the NGF protein comprises or preferably consists of an amino acid sequence, in which SEQ ID NO:22 is altered by at least one and by at most 20, preferably by at most 10, preferably by at most 5 amino acids by deletion. In one further preferred embodiment, the NGF protein comprises or preferably consists of an amino acid sequence, in which SEQ ID NO:22 is altered by one, two or three amino acids deletion.

In a further preferred embodiment, the NGF protein comprises or preferably consists of a mutated amino acid sequence, wherein the amino acid sequence to be mutated is SEQ ID NO:22, and wherein at most 10, 9, 8 7, 6, 5, or 4 amino acid residues are deleted from said amino acid sequence to be mutated. In a very preferred embodiment, the NGF protein comprises or preferably consists of a mutated amino acid sequence, wherein the amino acid sequence to be mutated is SEQ ID NO:22, and wherein 3, 2, or, preferably, 1 amino acid residue(s) are deleted from said amino acid sequence to be mutated.

In one preferred embodiment, the NGF protein comprises or preferably consists of an amino acid sequence, in which SEQ ID NO:22 is altered by at least one and by at most 20, preferably by at least one and by at most 10, more preferably by at least one and by at most 5 amino acids by insertion. In one further preferred embodiment, the NGF protein comprises or preferably consists of an amino acid sequence, in which SEQ ID NO:22 is altered by one, two or three amino acid insertions.

In a further preferred embodiment, the NGF protein comprises or preferably consists of a mutated amino acid sequence, wherein the amino acid sequence to be mutated is SEQ ID NO:22, and wherein at most 20, 19, 18, 17, 16, 15, 14, 13, 12, 11, 10, 9, 8 7, 6, 5, or 4 amino acid residues are inserted into said amino acid sequence to be mutated. In a very preferred embodiment, the NGF protein comprises or preferably consists of a mutated amino acid sequence, wherein the amino acid sequence to be mutated is SEQ ID NO:22, and wherein 3, 2, or, preferably, 1 amino acid residue(s) are inserted into said amino acid sequence to be mutated.

In one preferred embodiment, the NGF protein comprises or preferably consists of an amino acid sequence, in which SEQ ID NO:22 is altered by at least one and by at most 20, preferably by at least one and by at most 10, more preferably by at least one and by at most 5 amino acids by substitution, preferably by conservative substitution.

In one further preferred embodiment, the NGF protein comprises or preferably consists of an amino acid sequence, in which SEQ ID NO:22 is altered by one, two, three, four, five, six, seven, eight, nine or ten amino acids substitution(s), preferably by conservative substitution(s). In one further preferred embodiment, the NGF protein comprises or preferably consists of an amino acid sequence, in which SEQ ID NO:22 is altered by one, two or three amino acids substitution(s), preferably by conservative substitution(s).

In a further preferred embodiment, the NGF protein comprises or preferably consists of a mutated amino acid sequence, wherein the amino acid sequence to be mutated is SEQ ID NO:22, and wherein at most 20, 19, 18, 17, 16, 15, 14, 13, 12, 11, 10, 9, 8 7, 6, 5, or 4 amino acid residues of said amino acid sequence to be mutated are exchanged by amino acid substitution, wherein preferably said amino acid substitution is a conservative amino acid substitution. In a very preferred embodiment, the NGF protein comprises or preferably consists of a mutated amino acid sequence, wherein the amino acid sequence to be mutated is SEQ ID NO:22, and wherein 3, 2, or, preferably, 1 amino acid residue(s) of said amino acid sequence to be mutated are exchanged by amino acid substitution, wherein preferably said amino acid substitution is a conservative amino acid substitution.

In one preferred embodiment, the NGF protein is the human NGF precursor. In one further preferred embodiment, the human NGF precursor comprises or preferably consists of the amino acid sequence set forth in SEQ ID NO:23. In another preferred embodiment, the NGF protein comprises or preferably consists of an amino acid sequence, which is at least 80%, preferably at least 85%, more preferably at least 90%, and most preferably at least 95% identical with SEQ ID NO:23.

In one preferred embodiment, the NGF antigen is an NGF fragment, wherein said NGF fragment comprises or alternatively consists of at least one epitope. Methods to determine the epitope(s) of a protein are known to the artisan. PCT/EP2005/004980 has elaborated some of these methods from the first paragraph of page 26 to the fourth paragraph of page 27 therein, and these specific disclosures are incorporated herein by reference. It is to be noted that these methods are generally applicable to other polypeptide antigens, and therefore are not restricted to IL-23 p19 as disclosed in PCT/EP2005/004980.

In a preferred embodiment of the present invention, the NGF antigen is an NGF fragment. In a further preferred embodiment said NGF fragment comprises, or alternatively or preferably consists of, at least 8, preferably at least 12, more preferably at least 20, still more preferably at least 30 contiguous amino acids of the human NGF of SEQ ID NO:22. In a further preferred embodiment said NGF fragment consists of 5, 6, 7, 8, 9, 10, 11, 12, 13, 14, 15, 16, 17, 18, 19, 20, 21, 22, 23, 24, 25, 26, 27, 28, 29, or 30 contiguous amino acids of SEQ ID NO:22, wherein preferably said NGF fragment consists of 10 contiguous amino acids of SEQ ID NO:22, and wherein further preferably said NGF fragment consists of the 10 N-terminal amino acids of SEQ ID NO:22. Thus, in a very preferred embodiment said NGF fragment consists of the amino acid sequence of SEQ ID NO:44.

In one preferred embodiment, the NGF antigen is a NGF mutein, wherein preferably said NGF mutein comprises reduced biological activity, and wherein further preferably said NGF mutein is capable of inducing neutralizing antibodies when administered to an animal in form of an immunogenic conjugate. In a very preferred embodiment said NGF mutein does not comprises biological activity, and said NGF mutein is capable of inducing neutralizing antibodies when administered to an animal in form of an immunogenic conjugate.

In a further preferred embodiment said NGF mutein comprises or preferably consists of a mutated amino acid sequence, wherein the amino acid sequence to be mutated is SEQ ID NO:22, and wherein said amino acid sequence to be mutated is altered in at least one and in at most 9, preferably in at least one and in at most 3, more preferably in at least one and in at most 2 positions, most preferably in exactly one position, wherein the amino acid residue(s) in said position(s) are/is altered by substitution or by deletion, most preferably by substitution.

In a further preferred embodiment said NGF mutein consists of a mutated amino acid sequence, wherein the amino acid sequence to be mutated is SEQ ID NO:22, and wherein said amino acid sequence to be mutated is altered in at least one and in at most 9 positions, wherein the amino acid residue(s) in said position(s) are deleted from SEQ ID NO:22, and wherein preferably said deleted amino acid residues are selected from the amino acid residues 1 to 9 of SEQ ID NO:22, and wherein further preferably said deleted amino acid residues are selected from any one of the amino acid residues 4 H, 5 P, 7 F or 8 H of SEQ ID NO:22 (Kullander et al. J Biol Chem, 1997. 272(14): p. 9300-7; Woo, S. B. and K. E. Neet J Biol Chem, 1996. 271(40): p. 24433-41; and Beglova, N., et al. J Biol Chem, 1998. 273(37): p. 23652-8). In a very preferred embodiment, said NGF mutein consists of SEQ ID NO:45.)

In a further preferred embodiment said NGF mutein comprises or preferably consists of a mutated amino acid sequence, wherein the amino acid sequence to be mutated is SEQ ID NO:22, and wherein said amino acid sequence to be mutated is altered in at least one and in at most 3, preferably in at least one and in at most 2, and most preferably in exactly one position, wherein the amino acid residue(s) in said position(s) are/is altered by substitution.

In a further preferred embodiment said NGF mutein comprises or preferably consists of a mutated amino acid sequence, wherein the amino acid sequence to be mutated is SEQ ID NO:22, and wherein said amino acid sequence to be mutated is altered in at least one and in at most 3, preferably in at least one and in at most 2, and most preferably in exactly one position, wherein the amino acid residue(s) in said position(s) are/is altered by substitution, wherein the substituted amino acid residue(s) are selected from any one of the amino acid residues 4 H, 5 P, 7 F or 8 H of SEQ ID NO:22 (Kullander et al. J Biol Chem, 1997. 272(14): p. 9300-7; Woo, S. B. and K. E. Neet J Biol Chem, 1996. 271(40): p. 24433-41; and Beglova, N., et al. J Biol Chem, 1998. 273(37): p. 23652-8.). In a very preferred embodiment, said NGF mutein consists of any one of SEQ ID NOs 46 to 52.

In a further preferred embodiment said NGF mutein comprises or preferably consists of a mutated amino acid sequence, wherein the amino acid sequence to be mutated is SEQ ID NO:22, and wherein said amino acid sequence to be mutated is altered in at least one and in at most 3, preferably in at least one and in at most 2, and most preferably in exactly one position, wherein the amino acid residue(s) in said position(s) are/is altered by substitution, wherein the substituted amino acid residue(s) are selected from any one of the amino acid residues 43 to 45, 48, and 49 of SEQ ID NO:22 (Kullander et al. J Biol Chem, 1997. 272(14): p. 9300-7; Beglova, N., et al. J Biol Chem, 1998. 273(37): p. 23652-8, and Xie, Y., et al. J Biol Chem, 2000. 275(38): p. 29868-74.). In a very preferred embodiment, said NGF mutein consists of any one of SEQ ID NOs 53 to 60.

In a further preferred embodiment said NGF mutein comprises or preferably consists of a mutated amino acid sequence, wherein the amino acid sequence to be mutated is SEQ ID NO:22, and wherein said amino acid sequence to be mutated is altered in at least one and in at most 3, preferably in at least one and in at most 2, and most preferably in exactly one position, wherein the amino acid residue(s) in said position(s) are/is altered by substitution, wherein the substituted amino acid residue(s) are selected from any one of the amino acid residues 94 G, 95 K and 96 Q of SEQ ID NO:22 (Kullander et al. J Biol Chem, 1997. 272(14): p. 9300-7; Beglova, N., et al. J Biol Chem, 1998. 273(37): p. 23652-8, and Xie, Y., et al. J Biol Chem, 2000. 275(38): p. 29868-74.). In a very preferred embodiment, said NGF mutein consists of any one of SEQ ID NOs 61 to 66.

In a further preferred embodiment said NGF mutein comprises or preferably consists of a mutated amino acid sequence, wherein the amino acid sequence to be mutated is SEQ ID NO:22, and wherein said amino acid sequence to be mutated is altered in exactly one position by substitution, wherein the substituted amino acid residue is selected from any one of the amino acid residues 75 H, 84 H, 100 R, 111 V, 112 L, 114 R, 115 K, 113 S (Kullander, K. et al. J Biol Chem, 1997. 272(14): p. 9300-7; Larsson, E. et al. Neurobiol Dis, 2008, Kruttgen, A., et al. J Biol Chem, 1997. 272(46): p. 29222-8). In a very preferred embodiment, said NGF mutein consists of any one of SEQ ID NOs 67 to 74.

In a further preferred embodiment said NGF mutein comprises or preferably consists of a mutated amino acid sequence, wherein the amino acid sequence to be mutated is SEQ ID NO:22, and wherein said amino acid sequence to be mutated is altered in exactly 2 positions, wherein the amino acid residue(s) in said position(s) are altered by substitution, wherein the substituted amino acid residue(s) are 75 H and 84 H (Kullander et al. J Biol Chem, 1997. 272(14): p. 9300-7). In a very preferred embodiment, said NGF mutein consists of SEQ ID NO:75.

In one preferred embodiment, the composition comprises or alternatively consists essentially of, or alternatively consists of (a) a virus-like particle with at least one first attachment site; and (b) at least one antigen with at least one second attachment site, wherein said at least one antigen is a NGF antigen; and wherein (a) and (b) are linked through said at least one first and said at least one second attachment site, and wherein said linkage is via at least one peptide bond, preferably exclusively via peptide bond(s). In a further preferred embodiment said virus-like particle with at least one first attachment site and said at least one antigen are linked by way of genetic fusion. A gene encoding NGF antigen is in-frame ligated, either internally or preferably to the N- or the C-terminus of the gene encoding the coat protein of the VLP. Fusion may also be effected by inserting sequences of the NGF antigen into a mutant of a coat protein where part of the coat protein sequence has been deleted, that are further referred to as truncation mutants. Truncation mutants may have N- or C-terminal, or internal deletions of part of the sequence of the coat protein. The fusion protein shall preferably retain the ability of assembly into a VLP upon expression which can be examined by electron microscopy.

Flanking amino acid residues may be added to the NGF antigen in order to increase the distance between the coat protein and the NGF antigen. Glycine and serine residues are particularly favored amino acid residues to be used in the flanking sequences. Such flanking sequences confer additional flexibility to the fusion construct. This diminishes the potential destabilizing effect the foreign sequence which is fused into the sequence of a VLP subunit, and, thus, this diminishes the interference of the foreign sequence with the VLP assembly.

In other embodiments, the NGF antigen can be fused to a number of other viral coat proteins, by way of example, to the C-terminus of a truncated form of the Al protein of Qβ (Kozlovska, T. M., et al., Intervirology 39:9-15 (1996)). Alternatively, NGF antigen may be inserted between position 72 and 73 of the CP extension. For example, Kozlovska et al., (Intervirology, 39: 9-15 (1996)) describe QβA1 protein fusions where the epitope is fused at the C-terminus of the QβCP extension truncated at position 19. As another example, the NGF antigen can be inserted between amino acid 2 and 3 of the fr CP (Pushko P. et al., Prot. Eng. 6:883-891 (1993)). Furthermore, the NGF antigen can be fused to the N-terminal protuberant β-hairpin of the coat protein of RNA-bacteriophage MS-2 (WO 92/13081). Alternatively, the NGF antigen can be fused to a coat protein of papillomavirus, preferably to the major coat protein Ll of bovine papillomavirus type 1 (BPV-1) (Chackerian, B. et al., Proc. Natl. Acad. Sci. USA 96:2373-2378 (1999), WO 00/23955). Substitution of amino acids 130-136 of BPV-1 L1 with an NGF antigen is also an embodiment of the invention. Further embodiments of fusing NGF antigen to coat protein of a virus, or to mutants or fragments thereof, have been disclosed in WO 2004/009124 page 62 line 20 to page 68 line 17 and herein are incorporated by way of reference.

In another preferred embodiment, the NGF antigen is fused to either the N- or the C-terminus of a coat protein, or of mutants or fragments thereof, of RNA-bacteriophage AP205, wherein preferably said coat protein, or of mutants or fragments thereof comprises or preferably consists of the amino acid sequence selected from any one of SEQ ID NOs 14, and 40 to 42, most preferably of SEQ ID NO:41.

In one further preferred embodiment, the fusion protein further comprises a spacer, wherein said spacer is positioned between said coat protein, or mutant or fragment thereof, of RNA-bacteriophage AP205 and said NGF antigen. Preferably, said spacer is composed of less than 20, 19, 18, 17, 16, 15, 14, 13, 12, 11, 10, 9, 8, 7, 6, or 5 amino acids. Very preferably, said spacer is composed of 1, 2, 3, 4, 5, 6, 7, 8, 9, 10, 11, 12, 13, 14, or 15 amino acids.

In one preferred embodiment, the composition comprises or alternatively consists essentially of, or alternatively consists of (a) a virus-like particle with at least one first attachment site; and (b) at least one antigen with at least one second attachment site, wherein said at least one antigen is a NGF antigen; and wherein (a) and (b) are linked through said at least one first and said at least one second attachment site, and wherein said linkage is via at least one non-peptide covalent bond, and wherein preferably said first attachment site does not comprise or is not a sulfhydryl group, and wherein still more preferably said first attachment site does not comprise or is not a sulfhydryl group of a cysteine.

In one preferred embodiment, the first attachment site comprises, or preferably is, an amino group, preferably the amino group of a lysine residue. In a further preferred embodiment, said first attachment site comprises, or preferably is, an amino group of a lysine residue, wherein said lysine residue is a lysine residue of a recombinant coat protein comprised by said VLP. In a further preferred embodiment, said first attachment site comprises, or preferably is, an amino group of a lysine residue, wherein said lysine residue is a lysine residue of a recombinant coat protein of an RNA bacteriophage which is comprised by said VLP.

In a further preferred embodiment said virus-like particle with said at least one first attachment site is a virus-like particle of an RNA-bacteriophage, preferably of RNA-bacteriophage Qβ, and said first attachment site comprises, or preferably is, an amino group of a lysine residue, wherein preferably said lysine residue is a lysine residue of a recombinant coat protein, preferably of a recombinant coat protein of RNA-bacteriophage Qβ, wherein said recombinant coat protein is comprised by said virus-like particle of an RNA-bacteriophage.

In a further preferred embodiment said virus-like particle with at least one first attachment site comprises, consists essentially of, or alternatively consists of recombinant coat proteins, mutants or fragments thereof, of an RNA-bacteriophage, preferably of RNA-bacteriophage Qβ, and said first attachment site comprises, or preferably is, an amino group of a lysine residue, wherein preferably said lysine residue is a lysine residue contained in said recombinant coat protein, mutant or fragments thereof, of said RNA-bacteriophage, preferably of RNA-bacteriophage Qβ.

In a further preferred embodiment said virus-like particle with at least one first attachment site comprises, consists essentially of, or alternatively consists of a recombinant coat protein of an RNA-bacteriophage, wherein said recombinant coat proteins comprise or preferably consist of the amino acid sequence of SEQ ID NO:1; and wherein said first attachment site comprises, or preferably is, an amino group of a lysine residue of SEQ ID NO:1.

In another preferred embodiment of the present invention, the second attachment site comprises, or preferably is, a sulfhydryl group, preferably a sulfhydryl group of a cysteine residue. In a very preferred embodiment of the invention, the at least one first attachment site is an amino group, preferably an amino group of a lysine residue, and the at least one second attachment site is a sulfhydryl group, preferably a sulfhydryl group of a cysteine residue.

In one preferred embodiment of the invention, the NGF antigen is linked to the VLP by way of chemical cross-linking, typically and preferably by using a hetero-bifunctional cross-linker In preferred embodiments, the hetero-bifunctional cross-linker contains a functional group which can react with the preferred first attachment sites, preferably with the amino group, more preferably with the amino groups of lysine residue(s) of the VLP, and a further functional group which can react with the preferred second attachment site, i.e. a sulfhydryl group, preferably of cysteine(s) residue inherent of, or artificially added to the NGF antigen, and optionally also made available for reaction by reduction. Several hetero-bifunctional cross-linkers are known in the art. These include the preferred cross-linkers SMPH (Pierce), Sulfo-MBS, Sulfo-EMCS, Sulfo-GMBS, Sulfo-SIAB, Sulfo-SMPB, Sulfo-SMCC, SVSB, SIA and other cross-linkers available for example from the Pierce Chemical Company, and having one functional group reactive towards amino groups and one functional group reactive towards sulfhydryl groups. The above mentioned cross-linkers all lead to formation of an amide bond after reaction with the amino group and a thioether linkage with the sulfhydryl groups. Another class of cross-linkers suitable in the practice of the invention is characterized by the introduction of a disulfide linkage between the NGF antigen and the VLP upon coupling. Preferred cross-linkers belonging to this class include, for example, SPDP and Sulfo-LC-SPDP (Pierce).

In a preferred embodiment, the composition of the invention further comprises a linker. Engineering of a second attachment site to the NGF antigen is achieved by the association of a linker, preferably containing at least one amino acid suitable as second attachment site according to the disclosures of this invention. Therefore, in a preferred embodiment of the present invention, a linker is associated to the NGF antigen by way of at least one covalent bond, preferably, by at least one, typically one peptide bond. Preferably, the linker comprises, or alternatively consists of, the second attachment site. In a further preferred embodiment, the linker comprises a sulfhydryl group, preferably of a cysteine residue. In another preferred embodiment, the amino acid linker is a cysteine residue. In a further preferred embodiment, the amino acid linker is a CGG- or a GCG-linker, preferably a CGG-linker. Linkers which are suitable for the purposes of the invention are disclosed in WO2005/108425A1, page 32-33, which is incorporated herein by way of reference.

Linking of the NGF antigen to the VLP by using a hetero-bifunctional cross-linker according to the preferred methods described above, allows coupling of the NGF antigen to the VLP in an oriented fashion. Other methods of linking the NGF antigen to the VLP include methods wherein the NGF antigen is cross-linked to the VLP, using the carbodiimide EDC, and NHS. The NGF antigen may also be first thiolated through reaction, for example with SATA, SATP or iminothiolane. The NGF antigen, after deprotection if required, may then be coupled to the VLP as follows. After separation of the excess thiolation reagent, the NGF antigen is reacted with the VLP, previously activated with a hetero-bifunctional cross-linker comprising a cysteine reactive moiety, and therefore displaying at least one or several functional groups reactive towards cysteine residues, to which the thiolated NGF antigen can react, such as described above. Optionally, low amounts of a reducing agent are included in the reaction mixture. In further methods, the NGF antigen is attached to the VLP, using a homo-bifunctional cross-linker such as glutaraldehyde, DSG, BM[PEO]4, BS3, (Pierce) or other known homo-bifunctional cross-linkers with functional groups reactive towards amine groups or carboxyl groups of the VLP.

In other embodiments of the present invention, the composition comprises or alternatively consists essentially of a virus-like particle linked to NGF antigen via chemical interactions, wherein at least one of these interactions is not a covalent bond. Such interactions include but not limited to antigen-antibody interaction, receptor-ligand interaction Linking of the VLP to the NGF antigen can be effected by biotinylating the VLP and expressing the NGF antigen as a streptavidin-fusion protein.

One or several antigen molecules, i.e. NGF antigen, can be attached to one subunit of the VLP, preferably of coat proteins of RNA-bacteriophages, preferably through the exposed lysine residues of the coat proteins of VLPs of RNA-bacteriophages, if sterically allowable. A specific feature of the VLPs of RNA-bacteriophages and in particular of the VLP of RNA-bacteriophage Qβ, is thus the possibility to couple several antigens per subunit. This allows for the generation of a dense antigen array.

In a very preferred embodiment of the invention, the NGF antigen is linked via a cysteine residue, having been added to either the N-terminus or the C-terminus of, or a natural cysteine residue within NGF antigen, to lysine residues of coat proteins of the VLPs of RNA-bacteriophages, and in particular to the coat protein of RNA-bacteriophage Qβ.

As described above, four lysine residues are exposed on the surface of the VLP of Qβ coat protein. Typically and preferably these residues are derivatized upon reaction with a cross-linker molecule. In the instance where not all of the exposed lysine residues can be coupled to an antigen, the lysine residues which have reacted with the cross-linker are left with a cross-linker molecule attached to the ε-amino group after the derivatization step. This leads to disappearance of one or several positive charges, which may be detrimental to the solubility and stability of the VLP. By replacing some of the lysine residues with arginines, as in the disclosed Qβ coat protein mutants, we prevent the excessive disappearance of positive charges since the arginine residues do not react with the preferred cross-linkers. Moreover, replacement of lysine residues by arginine residues may lead to more defined antigen arrays, as fewer sites are available for reaction to the antigen.

Accordingly, exposed lysine residues were replaced by arginines in the following Qβcoat protein mutants: Qβ-240 (Lys13-Arg; SEQ ID NO:15), Qβ-250 (Lys 2-Arg, Lys13-Arg; SEQ ID NO:17), Qβ-259 (Lys 2-Arg, Lys16-Arg; SEQ ID NO:19) and Qβ-251; (Lys16Arg, SEQ ID NO:18). In a further embodiment, we disclose a mutant coat protein of RNA-bacteriophage Qβ with one additional lysine residue Qβ-243 (Asn 10-Lys; SEQ ID NO:16), which is suitable for obtaining even higher density arrays of antigens than with wildtype coat protein of RNA-bacteriophage Qβ (SEQ ID NO:1).

In one preferred embodiment of the invention, said virus-like particle is recombinantly produced by a host, and wherein said virus-like particle is essentially free of host RNA, and wherein preferably said virus-like particle is essentially free of host nucleic acids, wherein preferably said virus-like particle is a virus-like particle of an RNA-bacteriophage. In a further preferred embodiment, the composition further comprises at least one polyanionic macromolecule bound to, preferably packaged into or enclosed in, the VLP.

In a preferred embodiment, said virus-like particle is a virus-like particle of an RNA-bacteriophage, preferably a virus-like particle of an RNA-bacteriophage Qβ, wherein said virus-like particle of an RNA-bacteriophage, preferably said virus-like particle of an RNA-bacteriophage Qβ, is recombinantly produced by a host, and wherein said virus-like particle of an RNA-bacteriophage, preferably said virus-like particle of an RNA-bacteriophage Qβ, is essentially free of host RNA, and wherein said composition further comprises at least one polyanionic macromolecule, wherein said at least one polyanionic macromolecule is packaged into said virus-like particle of an RNA-bacteriophage, preferably into said virus-like particle of an RNA-bacteriophage Qβ. In a very further preferred embodiment, said polyanionic macromolecule is polyglutamic acid and/or polyaspartic acid, preferably polyglutamic acid.

In this context, the term “essentially free of host RNA, preferably host nucleic acids” as used herein, refers to the amount of host RNA, preferably host nucleic acids, comprised by the VLP, which amount typically and preferably is less than 30 μg, preferably less than 20 μg, more preferably less than 10 μg, even more preferably less than 8 μg, even more preferably less than 6 μg, even more preferably less than 4 μg, most preferably less than 2 μg, per mg of the VLP. Most preferably, the host RNA, preferably the host nucleic acids, which are comprised by said VLP and/or by said composition of the invention are below the detection limit. Host, as used within the afore-mentioned context, refers to the host in which the VLP is recombinantly produced, wherein said host typically and preferably is E. coli. Conventional methods of determining the amount of RNA, preferably nucleic acids, are known to the artisan. The typical and preferred method to determine the amount of RNA, preferably nucleic acids, in accordance with the present invention is described in Example 17 of WO2006/037787A2. Identical, similar or analogous conditions are, typically and preferably, used for the determination of the amount of RNA, preferably nucleic acids, for inventive compositions comprising VLPs other than Qβ. The modifications of the conditions eventually needed are within the knowledge of the artisan. The numeric value of the amounts determined should typically and preferably be understood as comprising values having a deviation of ±10%, preferably having a deviation of ±5%, of the indicated numeric value.

Host RNA, preferably host nucleic acids: The term “host RNA, preferably host nucleic acids” refers to the RNA, or preferably nucleic acids, that are originally synthesized by the host. The RNA, preferably nucleic acids, may, however, undergo chemical and/or physical changes during the procedure of reducing or eliminating the amount of RNA, preferably nucleic acids, typically and preferably by way of the inventive methods, for example, the size of the RNA, preferably nucleic acids, may be shortened or the secondary structure thereof may be altered. The term host RNA or nucleic acids also includes these degradation products.

Reducing or eliminating the amount of host RNA, preferably host nucleic, minimizes or reduces unwanted T cell responses, such as inflammatory T cell response and cytotoxic T cell response, and other unwanted side effects, such as fever, while maintaining strong antibody response against the antigen.

In one aspect the invention relates to a process for producing the composition of the invention, wherein said process comprises the steps of: (a) providing a VLP with at least one first attachment site; (b) providing an NGF antigen with at least one second attachment site, and (c) combining said VLP and said NGF antigen to produce a composition, wherein said NGF antigen and said VLP are linked through the first and the second attachment sites. In a further preferred embodiment, the step of providing said VLP with at least one first attachment site further comprises the steps: (a) disassembling said virus-like particle, preferably said virus-like particle of an RNA-bacteriophage, to said coat proteins, mutants or fragments thereof, (b) purifying said coat proteins, mutants or fragments thereof; (c) reassembling said purified coat proteins, mutants or fragments thereof to a virus-like particle, wherein preferably said virus-like particle is essentially free of host RNA, preferably host nucleic acids. In a still further preferred embodiment, the reassembling of said purified coat proteins is effected in the presence of at least one polyanionic macromolecule. Methods for reassembling coat proteins of RNA-bacteriophages in the presence of at least one polyanionic macromolecule are, for example, disclosed in WO2006/037787A2.

In one aspect, the invention provides a vaccine composition comprising the composition of the invention.

In a further aspect, the invention provides a vaccine composition, wherein said vaccine composition comprises or consists of a therapeutically effective amount of any one of the compositions of the invention.

In one preferred embodiment, said vaccine composition further comprises at least one adjuvant. The administration of the at least one adjuvant may hereby occur prior to, contemporaneously or after the administration of the inventive composition. The term “adjuvant” as used herein refers to non-specific stimulators of the immune response or substances that allow generation of a depot in the host which when combined with the vaccine composition and pharmaceutical composition, respectively, of the present invention may provide for an even more enhanced immune response. Examples of the at least one adjuvant include and preferably consist of complete and incomplete Freund's adjuvant, aluminium hydroxide, aluminium salts, and modified muramyldipeptide. Further adjuvants are mineral gels such as aluminium hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art. Further adjuvants that can be administered with the compositions of the invention include, but are not limited to, Monophosphoryl lipid immunomodulator, AdjuVax 100a, QS-21, QS-18, CRL1005, Aluminium salts (Alum), MF-59, OM-174, OM-197, OM-294, and Virosomal adjuvant technology. Still further adjuvant include immunostimulatory nucleic acid, preferably the immunostimulatory nucleic acid contains one or more modifications in the backbone, preferably phosphorothioate modifications. The modification is to stabilize the nucleic acid against degradation.

In another preferred embodiment, the vaccine composition is devoid of adjuvant. An advantageous feature of the present invention is the high immunogenicity of the composition, even in the absence of adjuvants. Thus, the administration of the vaccine composition to a patient will preferably occur without administering at least one adjuvant to the same patient prior to, contemporaneously or after the administration of the vaccine composition. VLP has been generally described as an adjuvant. However, the term “adjuvant”, as used within the context of this application, refers to an adjuvant not being the VLP used for the inventive compositions, rather in addition to said VLP.

Vaccine compositions of the invention are said to be “pharmacologically acceptable” if their administration can be tolerated by a recipient individual. Further, the vaccine compositions of the invention will be administered in a “therapeutically effective amount”, i.e. an amount that produces a desired physiological effect. In the context of the invention, the desired physiological effect typically and preferably is the suppression or the reduction of pain.

In one aspect, the invention provides a pharmaceutical composition, wherein said pharmaceutical composition comprises a composition or a vaccine composition of the invention, together with a pharmaceutically acceptable carrier. In a preferred embodiment said pharmaceutical composition comprises a therapeutically effective amount of the composition of the invention. When a composition or a vaccine composition is administered to an individual, it may be in a form which contains salts, buffers, adjuvants, or other substances which are desirable for improving the efficacy of the conjugate. Examples of materials suitable for use in preparation of pharmaceutical compositions are provided in numerous sources including Remington's Pharmaceutical Sciences (Osol, A, ed., Mack Publishing Co., (1990)). Further components of pharmaceutical compositions include sterile aqueous (e.g., physiological saline) or non-aqueous solutions and suspensions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption.

The invention further discloses a method of immunization, preferably against NGF antigen, said method comprising administering the composition, the vaccine composition, or the pharmaceutical composition to an animal, preferably to a human. The animal is preferably a mammal, such as mouse, monkey, chimpanzee, dog, cat, horse and particularly human. The composition, the vaccine composition, or the pharmaceutical composition may be administered to said animal, preferably to said human by various methods known in the art. Typically and preferably, the composition, the vaccine composition, or the pharmaceutical composition is administered to said animal, preferably to said human, by injection, infusion, inhalation, or oral administration. Further preferably the composition, the vaccine composition, or the pharmaceutical composition is administered intramuscularly, intravenously, transmucosally, transdermally, intranasally, intraperitoneally or subcutaneously.

In one aspect, the present invention provides a method for preventing or in particular for treating pain, wherein said method comprises administering said composition, said vaccine composition or said pharmaceutical composition to an animal, preferably to a human, wherein typically and preferably said animal, and preferably said human, is suffering from pain. In one preferred embodiment, said pain is nociceptive pain. In a very preferred embodiment said pain is chronic inflammatory pain. In a further preferred embodiment said nociceptive pain is ostheoarthritic pain, rheumatoid arthritis pain, cancer pain, visceral pain, chronic low back pain, or chronic headache, pancreatitis pain, cystitis pain or prostatitis pain. In a very preferred embodiment said pain is bone cancer pain. In one preferred embodiment, the pain is caused by injury.

In one preferred embodiment, said pain is a neuropathic pain, which can be caused by, for example, nerve compression/nerve trauma due to injury, by infection of nerve cells, such as post-herpetic neuralgia, conditions leading to damage of nerve cells like stroke or degenerative neurological disorders or phantom limb pain.

A further aspect of the invention is the use of said composition, of said vaccine composition or of said pharmaceutical composition as a medicament.

In another aspect, the invention provides for the use of said composition or of said vaccine composition for the manufacture of a medicament for the treatment of pain in an animal, preferably in a human, wherein preferably said pain is nociceptive pain or neuropathic pain. In a very preferred embodiment said pain is chronic inflammatory pain. In a further preferred embodiment said pain is ostheoarthritic pain, rheumatoid arthritis pain, cancer pain, visceral pain, chronic low back pain, or chronic headache, pancreatitis pain, cystitis pain or prostatitis pain. In a very preferred embodiment said pain is bone cancer pain. In one preferred embodiment, the pain is caused by injury.

In another aspect, the invention provides a composition, a vaccine composition or a pharmaceutical composition as described herein for the treatment of pain in an animal, preferably in a human, wherein further preferably said composition, said vaccine or said pharmaceutical composition is to be administered to said animal, preferably to said human, and wherein still further preferably said pain is nociceptive pain or neuropathic pain.

In one embodiment, the present invention relates to a composition comprising, consisting essentially of, or preferably consisting of: (a) a virus-like particle (VLP) with at least one first attachment site, wherein said VLP is a VLP of RNA-bacteriophage Qβ, and (b) at least one antigen with at least one second attachment site, wherein said at least one antigen comprises an NGF protein and a linker, and wherein preferably said NGF protein consists of SEQ ID NO:22; and wherein (a) and (b) are linked through said at least one first and said at least one second attachment site, and wherein said first attachment site is an amino group of a lysine residue of said VLP of RNA-bacteriophage Qβ, and wherein said second attachment site is comprised by said linker, and wherein said linker is fused to said NGF protein by a peptide bond, and wherein said second attachment site is a sulfhydryl group of a cysteine; and wherein preferably said first attachment site and said second attachment site are linked through a hetero-bifunctional crosslinker, preferably by SMPH.

In one embodiment, the present invention relates to a composition comprising, consisting essentially of, or preferably consisting of: (a) a virus-like particle (VLP) with at least one first attachment site, wherein said VLP is a VLP of RNA-bacteriophage Qβ, and (b) at least one antigen with at least one second attachment site, wherein said at least one antigen comprises an NGF fragment and a linker, and wherein preferably said NGF fragment consists of SEQ ID NO:44; and wherein (a) and (b) are linked through said at least one first and said at least one second attachment site, and wherein said first attachment site is an amino group of a lysine residue of said VLP of RNA-bacteriophage Qβ, and wherein said second attachment site is comprised by said linker, and wherein said linker is fused to said NGF protein by a peptide bond, and wherein said second attachment site is a sulfhydryl group of a cysteine; and wherein preferably said first attachment site and said second attachment site are linked through a hetero-bifunctional crosslinker, preferably by SMPH.

In one embodiment, the present invention relates to a composition comprising, consisting essentially of, or preferably consisting of: (a) a virus-like particle (VLP) with at least one first attachment site, wherein said VLP is a VLP of RNA-bacteriophage Qβ, and (b) at least one antigen with at least one second attachment site, wherein said at least one antigen comprises an NGF mutein and a linker, and wherein preferably said NGF mutein consists of any one of SEQ ID NOs 45 to 75; and wherein (a) and (b) are linked through said at least one first and said at least one second attachment site, and wherein said first attachment site is an amino group of a lysine residue of said VLP of RNA-bacteriophage Qβ, and wherein said second attachment site is comprised by said linker, and wherein said linker is fused to said NGF protein by a peptide bond, and wherein said second attachment site is a sulfhydryl group of a cysteine; and wherein preferably said first attachment site and said second attachment site are linked through a hetero-bifunctional crosslinker, preferably by SMPH.

In one embodiment, the present invention relates to a composition comprising, consisting essentially of, or preferably consisting of: (a) a virus-like particle (VLP) with at least one first attachment site, wherein said VLP comprises or preferably consists of SEQ ID NO:1, and (b) at least one antigen with at least one second attachment site, wherein said at least one antigen comprises an NGF antigen and a linker, and wherein said NGF antigen is selected from the group consisting of (i) NGF protein, preferably SEQ ID NO:22; (ii) NGF fragment, preferably SEQ ID NO:44; and (iii) NGF mutein, preferably any one of SEQ ID NOs 45 to 75; and wherein (a) and (b) are linked through said at least one first and said at least one second attachment site, and wherein said first attachment site is an amino group of a lysine residue of said SEQ ID NO:1, and wherein said second attachment site is comprised by said linker, and wherein said linker is fused to said NGF antigen by a peptide bond, and wherein said second attachment site is a sulfhydryl group of a cysteine; and wherein preferably said first attachment site and said second attachment site are linked through a hetero-bifunctional crosslinker, preferably by SMPH.

In one embodiment, the present invention relates to a composition comprising, consisting essentially of, or preferably consisting of: (a) a virus-like particle (VLP) with at least one first attachment site, wherein said VLP is a VLP of RNA-bacteriophage Qβ, and (b) at least one antigen with at least one second attachment site, wherein said at least one antigen comprises an NGF antigen and a linker, and wherein said NGF antigen is selected from the group consisting of (i) NGF protein, preferably SEQ ID NO:22; (ii) NGF fragment, preferably SEQ ID NO:44; and (iii) NGF mutein, preferably any one of SEQ ID NOs 45 to 75; and wherein (a) and (b) are linked through said at least one first and said at least one second attachment site, and wherein said first attachment site is an amino group of a lysine residue of said VLP of RNA-bacteriophage Qβ, and wherein said second attachment site is comprised by said linker, and wherein said linker is fused to said NGF antigen by a peptide bond, and wherein said second attachment site is a sulfhydryl group of a cysteine; and wherein preferably said first attachment site and said second attachment site are linked through a hetero-bifunctional crosslinker, preferably by SMPH, wherein said composition further comprises at least one polyanionic macromolecule, wherein said polyanionic macromolecule is packaged into said VLP, and wherein preferably said polyanionic macromolecule is polyglutamic acid.

### EXAMPLE

### Example 1

### Cloning of mproNFGβ cDNA into a Prokaryotic Expression Vector

Mouse proNFGβ was amplified from a cDNA library of murine embryonal brain tissue by PCR using the following primers: Nde-proNGF-F (SEQ ID NO:26) and proNGF-Xho-R (SEQ ID NO:27). The PCR product was digested with NdeI and XhoI and ligated into pM-His-GGC vector. The resulting plasmid was named pM-proNGF-HisGGC, which encodes a fusion protein comprising mouse NGFβ3, a His₆ tag and a linker containing two glycines followed by a cysteine at the C-terminus.

### Example 2

### Construction of a pCB28_proNGFβ-His-GGC Eukaryotic Expression Vector

The mproNFGβ sequence was amplified from the pM-proNGF-HisGGC vector by PCR using the following two primers: mproNGFeuk_var1_f (SEQ ID NO: 28) and mproNGFeuk_var1_r (SEQ ID NO:29). The PCR product was ligated into pCRII-TOPO vector (Invitrogen). The pCRII-TOPO vector was digested with PacI and XhoI and the resulting mproNGFβ-HisGGC fragment ligated into the pCB28 vector. The resulting plasmid was named pCB28_mproNGF_His_C and encodes for a signal sequence derived of the kappa light chain of gamma immunoglobulin which was derived of the pSECTag2/Hygro A,B,C vector (Invitrogen) followed by the mproNFGβ sequence, a His₆ tag and a linker containing two glycines followed by a cysteine at the C-terminus.

### Example 3

### Expression of pCB28-proNGFβ-His-GGC in 293T HEK Cells

1 μg of the pCB28_proNGF_His_C expression vector containing the puromycin resistence gene was mixed with 200 μl Opti-MEM (Invitrogen) and 7.5 μl Lipofectamine 2000 (Invitrogen) and transfected into 1.5*10⁶ HEK 293T cells. After 4 hours cells were re-fed with D-MEM medium containing 10% FCS and 1% non-essential amino acids (Invitrogen). After 24 h, 1 mg/l puromycin (Invitrogen) was added to the medium to allow for selection for puromycin resistance. Puromycin resistant cells were grown in poly-L-lysine coated roller bottles in FCS-free D-MEM medium containing 10 mg/l glutathione reduced (Fluka), 161 mg/l N-acetyl-L-cyteine (Fluka) 1% non-essential amino acids, 1% penicillin/streptomycin (Invitrogen) and 1 mg/l puromycin.

Supernatants of puromycin resistant cells were harvested and concentrated 5-fold by Amicon ultra centrifugation filter devices (Millipore) and run on a 12% NUPAGE Bis/Tris gel (Invitrogen) under reducing conditions. Expression of NGF was visualized by immunoblot analysis using an anti penta histidine-specific antibody (Qiagen). Expression was demonstrated by visualization of a band of approximately 15 kDa which corresponds to the expected size of 14.7 kDa for mature mNGFβ containing a His-tag and a linker (SEQ ID NO:30). Correct processing of mature mNGFβ-HisGGC released into the supernatant from mproNGFβ-His-GGC-expressing HEK 293T cells was verified by Edman-degradation and revealed SSTHP as the five N-terminal amino acids of mature mNGFβ. This corresponded to the five expected amino acids of mature mNGFβ and verified correct expression and processing of mproNGFβ-His-GGC to mNGFβ-His-GGC by HEK 293T cells

### Example 4

### Purification of mNGFβ-His-GGC

mNGFβ-His-GGC was purified from HEK 293T supernatants by Ni²⁺-affinity purification. Briefly, supernatants were supplemented with 1/10^{th} volume of nickel-nitrilotriacetic acid (Ni-NTA)-binding buffer (500 mM NaH₂PO₄, 1.5 M NaCl, 100 mM Imidazol, 100 mM β-mercaptoethanol, pH8) and then pumped over equilibrated Ni-NTA superflow beads (Qiagen) packed into a column for 16 h at 4° C. using a peristaltic pump. After binding, the column was connected to an AEKTA Purifier FPLC System and Ni-NTA superflow beads were washed with four column volumes of washing buffer (50 mM NaH₂PO₄, 150 mM NaCl, 20 mM imidazol, pH8) to reduce the amount of unspecifically bound impurities. Subsequently mNGFβ was eluted with a gradient of 2 column volumes from washing to elution buffer (50 mM NaH₂PO₄, 150 mM NaCl, 500 mM imidazol, pH8). Eluted protein was run on a 12% NUPAGE Bis/Tris gel under reducing conditions to check the quality of the purification. Highly pure mNGFβ-His-GGC could be derived in such a way and was used for further development of the Qβ-mNGFβ-His-GGC as described in EXAMPLE 7.

### Example 5

### Recognition of mNGFβ-His-GGC by Monoclonal Antibodies

The binding of mNGFβ-His-GGC in comparison to natural tag and linker-free mNGFβ purified from submaxillary glands of mice was determined by sandwich ELISA using a monoclonal anti NGFβ antibody (Chemicon International) for capture and polyclonal sheep anti mNGFβ antibodies (Chemicon International) for detection. Briefly, monoclonal anti mNGFβ antibody was diluted to 0.85 μg/ml in carbonate buffer (0.1 M NaHCO₃, pH 9.6) and coated overnight at 4° C. on microtiter wells. Plates were then washed 3 times with PBS-0.05% Tween20 (PBS-T) and afterwards blocked with 2% BSA in PBS-T for 2 h at 37° C. Then plates were incubated for 2 h at room temperature with increasing concentrations of 0.2 to 200 ng/ml of mNGFβ-His-GGC or mNGFβ isolated from submaxillary glands diluted in 1% BSA in PBS-T. Then plates were washed 6 times with PBS-T and incubated with 10 μg/ml sheep anti mNGFβ antibody for 1 h at room temperature. After 6 washes with PBS-T, plates were incubated for 30 min at room temperature with 60 μg/ml peroxidase conjugated rabbit anti sheep antibody (Chemicon International). After six final washing steps with PBS-T the enzymatic reaction was started by adding 100 μl of a solution consisting of 10 mg OPD substrate (Fluka) and 8 μl H₂O₂ (30%) in 25 ml citrate buffer (66 mM NaH2PO4, 35 mM citric acid, pH5) to all wells. The colour reaction was stopped with 5% H₂SO₄ and absorbance measured at 450 nm using an ELISA reader (BioRad). The ELISA revealed equal recognition of both recombinantly expressed mNGFβ-His-GGC and mNFGβ indicating that mNGFβ-His-GGC is correctly expressed and folded in the correct tertiary conformation.

### Example 6

### Bioactivity of mNGFβ-His-GGC

The bioactivity of mNGFβ-His-GGC was tested in an in vitro cell proliferation assay using the mNGFβ-responsive factor dependent human erythroleukemic TF-1 cell line (ATCC) as readout system as described by others (R&D NGFβ product sheet). Briefly, 10⁴ TF-1 cells were seeded in 100 μl DMEM medium (supplemented with 10% FCS, 10 mM HEPES, 1% Penicillin/Streptomycin and 1% Glutamax) per well of a 96-well flat bottom plate. Increasing concentrations of 0.8 to 100 ng/ml of mNGFβ-HisGGC or mNGFβ purified from submaxilliary glands of mice was added to the cells. After 48 h cells were labelled with BrdU labelling reagent (Roche Diagnostics) to be incorporated into proliferating cells. 24 h later cells were fixed and subsequently incubated with a peroxidase conjugated anti-BrdU monoclonal antibody (Roche Diagnostics). After extensive washing, 100 μl of tetrabenzyl-benzidine substrate solution was given to each well. The colour reaction was stopped with 5% H₂SO₄ and absorbance measured at 450 nm using an ELISA reader (BioRad). Both mNGFβ-His-GGC and mNGFβ from submaxillary glands stimulated the proliferation in a similar fashion indicating that mNGFβ-His-GGC is biologically active.

### Example 7

### Coupling of mNGFβ-His-GGC to Qβ-VLP

Qβ-VLPs (2 mg/ml) were reacted with a 5-fold molar excess of the hetero-bifunctional cross-linker Succinimyl-6-(β-maleimidopropionamido)hexanoate (SMPH, Pierce) for 30 min at room temperature. SMPH was taken from a 50 mM stock dissolved in dimethyl sulfoxide. Reaction products were dialyzed against two changes of coupling buffer (20 mM MES, 300 mM NaCl, 10% glycerol, pH6) with a 10 kDa molecular weight cut-off (Slide-A-Lyzer, Pierce). Dialysis was performed at room temperature. Qβ-VLPs derivatized in such a way were then used for coupling to the target protein.

Before coupling, purified mNGFβ-His-GGC obtained as described in EXAMPLE 4 was dialyzed in coupling buffer and reduced for 1 h at room temperature (RT) with a 5-molar excess of TCEP. Reduced mNGFβ-His-GGC (0.25 mg/ml) was incubated with derivatized Qβ (0.25 mg/ml) in a total volume of 100 μl for 4 h at 4° C. After completion of the reaction the reaction tubes were centrifuged to remove possible precipitation. The coupling reaction was analysed by running the coupling product on a 12% NuPAGE Bis/Tris gel under reducing conditions and immunoblot analysis with an anti-penta His antibody and revealed efficient coupling of mNGFβ to Qβ-VLPs. Protein concentration was measured by Bradford.

### Example 8

### Immunogenicity of Qβ-mNGFβ-His-GGC

Male DBA/1 mice were immunized subcutaneously with 50 μg Qβ VLPs coupled to mNGFβ-His-GGC obtained from example 7 at day 0, day 10 and day 20 in the absence of adjuvant. As negative controls, mice were immunized with Qβ-VLPs only. At days 10, 20 and 30 blood was taken. Serum was prepared by spinning the blood samples in serum tubes (Mirotainer, BD Biosciences) at 10.000 g for 10 min. Detection of mNGFβ-specific antibodies in serum samples was done by ELISA using tag and linker free mNFGβ purified from submaxillary glands of mice for coating. Briefly, mNGFβ was diluted to a concentration of 2.5 μg/ml in carbonate buffer (0.1 M NaHCO₃, pH 9.6) and coated overnight at 4 ° C. on microtiter wells. Plates were then washed 3 times with PBS-0.05% Tween20 (PBS-T) and afterwards blocked with 2% BSA in PBS-T for 2 h at 37° C. Then plates were incubated for 2 h at room temperature with serum samples diluted in PBS-T+2% BSA using 3-fold dilution steps and starting with an initial dilution of 1:200. Then plates were washed 6 times with PBS-T and incubated with a 1:1000 dilution of a peroxidase conjugated goat anti mouse IgG-Fc antibody (Jackson ImmunoResearch Laboratories) for lh at room temperature. After 6 washes the enzymatic reaction was started by adding 100 μl of a solution consisting of 10 mg OPD substrate and 8 μl H₂O₂ (30%) in 25 ml citrate buffer (66 mM NaH2PO4, 35 mM citric acid, pH5) to all wells. The colour reaction was stopped with 5% H₂SO₄ and absorbance measured at 450 nm using an ELISA reader (BioRad). The ELISA revealed that strong autoantibody responses against mNFGβ could be induced by Qβ-mNGFβ-His-GGC using the immunisation protocol described above.

### Example 9

### Neutralizing Activity of Antibodies Raised with Qβ-mNGFβ-His-GGC

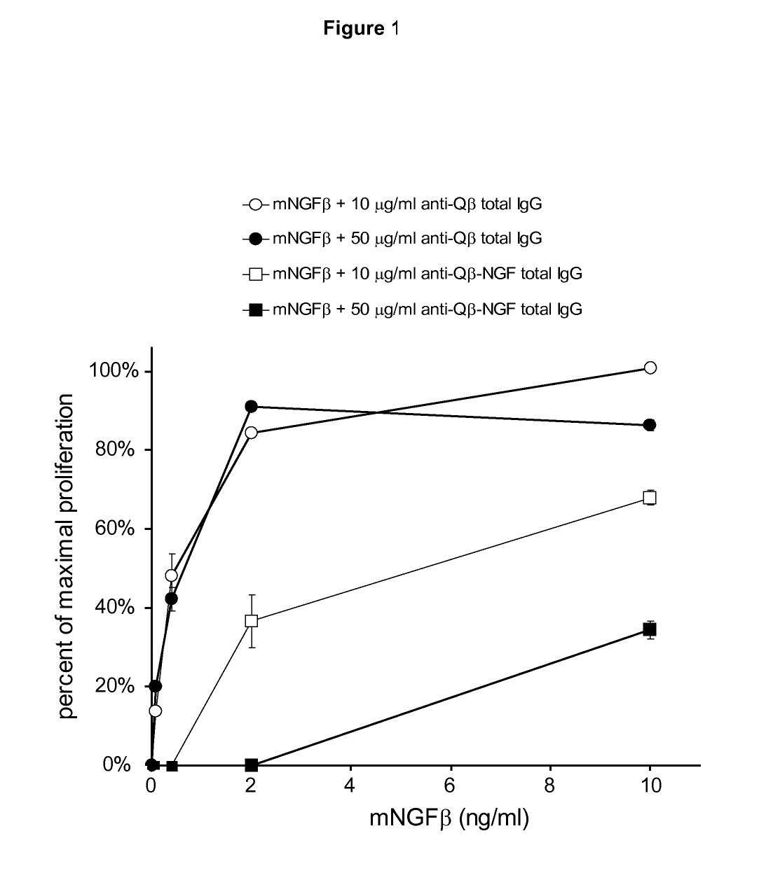

The in vitro neutralizing activity of antibodies raised by immunization with Qβ-mNGFβ-His-GGC was tested in an in vitro cell proliferation assay as described in EXAMPLE 6. Briefly, mice were immunized with either Qβ or Qβ-mNGFβ-His-GGC as described in EXAMPLE 8. Sera of immunized animals were collected and total IgG fractions isolated from sera by protein G purification. The capacity of purified total IgGs from Qβ or Qβ-mNGFβ-His-GGC immunized mice to neutralize the bioactivity of mNGFβ was then tested in the TF-1 cell proliferation assay. Briefly, 10⁴ TF-1 cells were seeded in 100 μl DMEM medium (supplemented with 10% FCS, 10 mM HEPES, 1% Penicillin/Streptomycin and 1% Glutamax) per well of a 96-well flat bottom plate. Increasing concentrations of 0.2 to 10 ng/ml of mNGFβ purified from submaxilliary glands were pre-incubated with total IgGs purified from sera of mice immunized with Qβ or Qβ-mNGFβ-His-GGC for 30 min at RT and then added to the cells. After 48 h cells were labeled with BrdU labelling reagent (Roche Diagnostics) to be incorporated into proliferating cells. 24 h later cells were fixed and subsequently incubated with a peroxidase conjugated anti-BrdU monoclonal antibody (Roche Diagnostics). After extensive washing, 100 μl of tetrabenzyl-benzidine substrate solution was given to each well. The color reaction was stopped with 5% H₂SO₄ and absorbance measured at 450 nm using an ELISA reader (BioRad). Cells stimulated with increasing concentrations of mNGFβ pre-incubated with 10 μg/ml or 50 μg/ml total IgGs from Qβ immunized mice showed no suppression of proliferation. Cells stimulated with increasing concentrations of mNGFβ pre-incubated with 10 μg/ml or 50 μg/ml total IgGs from Qβ-mNGFβ-His-GGC on the other hand exhibited significantly decreased proliferation rates proving the NGF-neutralizing activity of antibodies raised by immunization with Qβ-mNGFβ-His-GGC (FIG. 1).

### Example 10

### Efficacy of Qβ-mNGF-His-GGC Vaccination in Suppressing Cachexia in Collagen Induced Arthritis in Mice

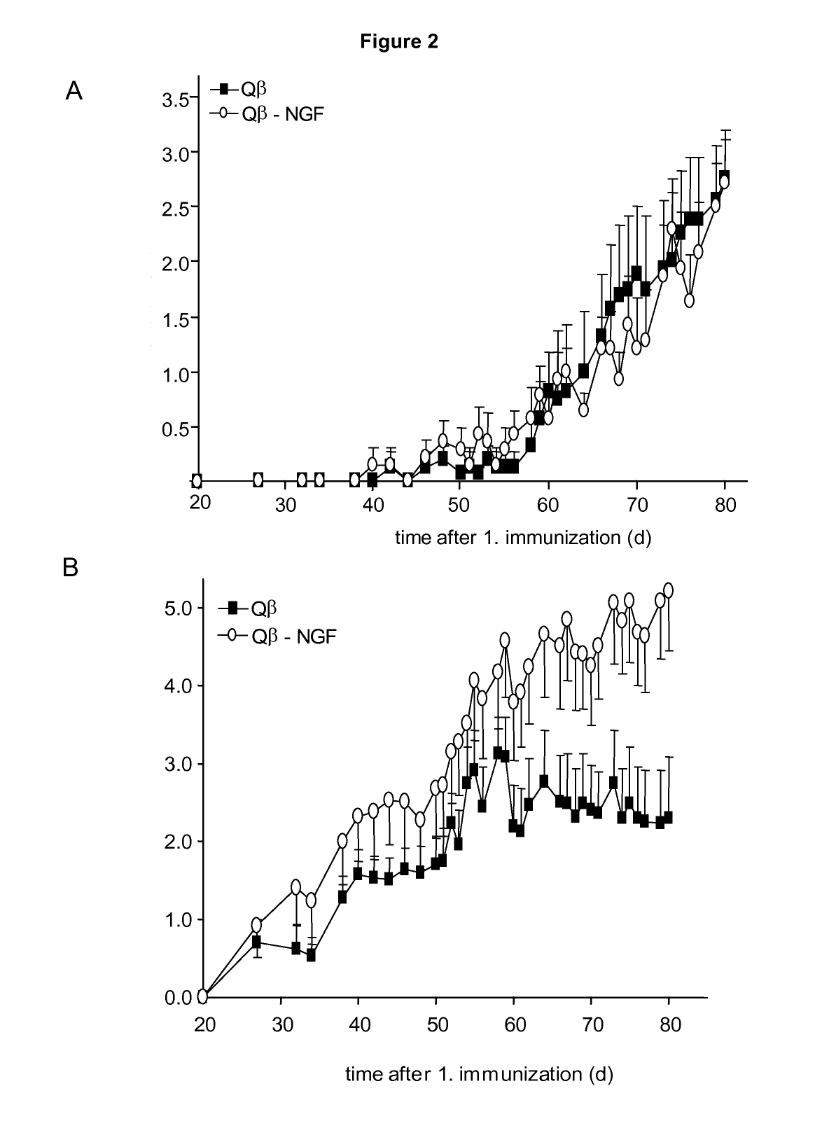

The ability of the Qβ-mNGF-His-GGC vaccine to reduce cachexia in autoimmune arthritis was evaluated in a mouse model of Rheumatoid Arthritis (RA), so called Collagen Induced Arthritis (CIA). In this model RA was induced by intradermal injection of collagen type 2 (MD Biosciences) in Complete Freunds Adjuvant (CFA) followed by an intradermal injection of collagen type II in Incomplete Freunds Adjuvant (IFA) 21 days later. The inflammation progresses steadily and culminates in ankylosis and permanent joint destruction accompanied by weight loss. Male DBA/1 mice (n=8) were immunized with 50 μg Qβ-mNGF-His-GGC at day −35, day −21 and day −7, as a negative control mice were immunised only with Qβ. After three immunizations, RA was induced at day 0. The inflammatory process was monitored over 7-9 weeks and clinical scores were assigned to each limb according to the following definitions: 0 normal, 1 mild erythema and/or swelling of digits/paw, 2 erythema and swelling extending over whole paw/joint, 3 strong swelling, deformation of paw/joint, with ankylosis. FIG. 2A shows the progression of arthritis in both the Qβ and Qβ-mNGF-His-GGC immunized animals. Arthritis progressed to a very similar extend in both groups. During the same period the body weight of mice was determined on a daily basis (FIG. 2B) and showed a substantial deviation in the average body weight between both groups especially after onset of the disease indicating a suppression of autoimmunity associated cachexia in Qβ-mNGF-His-GGC immunized mice.

### Example 11

### Efficacy of Qβ-mNGFβ-His-GGC Vaccination in Suppressing Inflammatory Pain in a Zymosan A-Induced Inflammatory Pain Model in Mice

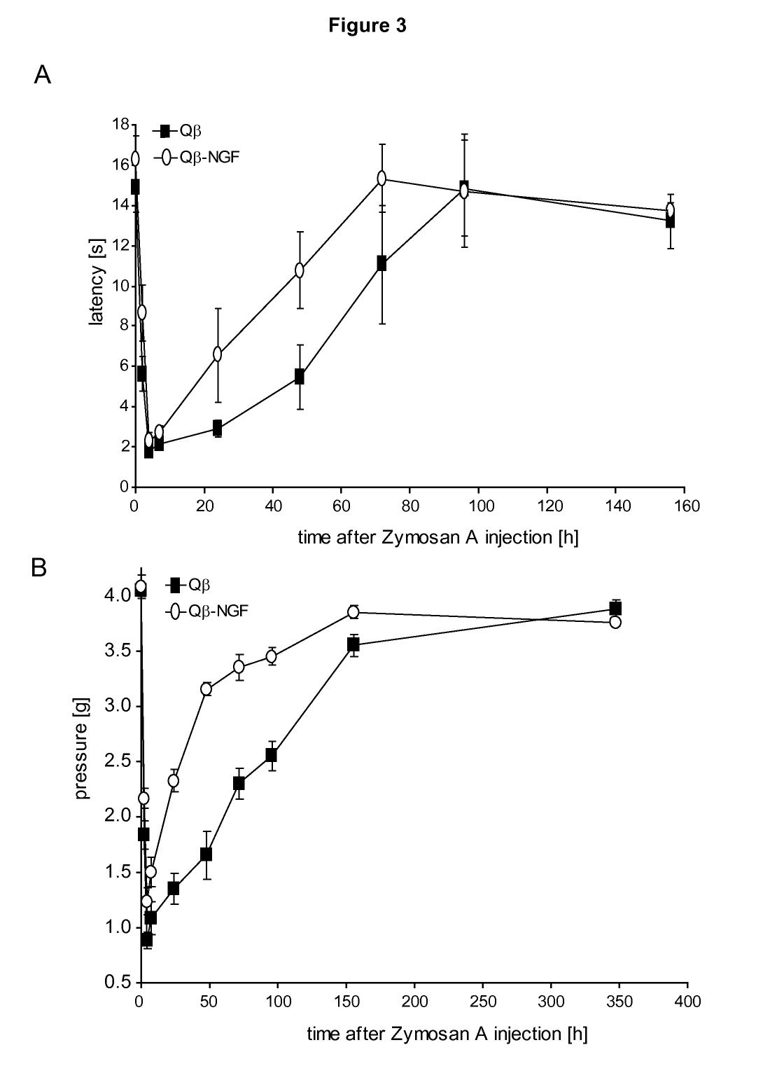

The ability of the Qβ-mNGF-His-GGC vaccine to reduce inflammatory hypersensitivity in response to thermal and mechanical stimulation caused by the injection of the yeast extract zymosan A (Sigma) into the plantar side of hind feet of male BL/6 mice was evaluated in the following way: Male BL/6 mice (n=4) were immunized at days 0, 10 and 20 with 50 μg of Qβ-mNGFβ-His-GGC or Qβ alone. At each time point thermal hypersensitivity of the plantar side of both hind feet was determined in response to thermal and mechanical stimulation. For determination of thermal sensitivity latency of foot withdrawal after stimulation with a heat source of defined intensity (infrared beam) was measured. Latency was determined with an electronically controlled instrument (Plantartest, Ugo Basile). The intensity of the heat source was adjusted so that for untreated animals latency was approximately 16 s. For each hind foot 6 measurements were taken and mean values calculated. To determine mechanical sensitivity, the response to stimulation with so called Von Frey Filaments was measured. Von Frey Filaments are calibrated plastic filaments with which an increasing pressure can be applied to the plantar side of the mouse foot. With an electronically controlled instrument (IITC, Woodland Hills, USA) the applied pressure which led to foot withdrawal was determined. Six measurements were taken per foot and mean values calculated. At day 31 inflammatory pain was induced by injection of 20 μl of a 3 mg/ml solution of zymosan A into the plantar side of the left hind foot. Hypersensitivity in response to thermal and mechanical stimulation which arises shortly after onset of the zymosan A induced inflammation was determined as described above by paw withdrawal 2 h, 4 h, 6 h, 8 h, 1 d, 2 d, 3 d, 4 d and 7 d after induction of the inflammatory pain. As demonstrated in FIG. 3, animals that were immunized with Qβ-mNGF-His-GGC showed a significantly reduced hypersensitivity after both thermal (FIG. 3A) and mechanical stimulation (FIG. 3B) as compared to animals immunized with Qβ only.

### Example 12

### Efficacy of NGF-His-GGC in Suppressing Neuropathic Pain in a Chronic Constriction Injury-Induced Neuropathic Pain Model in Mice

Male BL/6 mice (n=6) are immunized at days 0, 14 and 28 with 50 μg of Qβ-mNGFβ-His-GGC or Qβ alone. At each time point thermal hypersensitivity of the plantar side of both hind feet is determined in response to thermal and mechanical stimulation as described in EXAMPLE 11. At day 42 chronic constriction injury (CCI) is induced by ligating the sciatic nerve or leaving it unaffected (sham operated control group). Ligation of the sciatic nerve causes hypersensitivity in the respective limb which develops within 14 days and lasts for another 2-3 weeks before it declines. CCI is an established rodent model for neuropathic pain. After the CCI operation, sensitivity in response to thermal and mechanical stimulation is measured every other day as described in EXAMPLE 11.

### Example 13

### Cloning, Expression, Purification and Coupling of Human NGFβ to Qβ-VLPs

Human proNFGβ is amplified from a human fetal brain tissue cDNA library by PCR and the PCR product is ligated into the pCB28 expression vector. The resulting plasmid is named pCB28_huproNGF_His_C and encodes for a signal sequence derived of the kappa light chain of gamma immunoglobulin followed by the huproNFGβ sequence a His₆ tag and a linker containing two glycines followed by a cysteine at the C-terminus. The eukaryotic expression vector pCB28_huproNGF_His_C is transfected into HEK 293T cells essentially as described in EXAMPLE 3 and supernatants containing mature huNGFβ-His-GGC are harvested for purification by Ni-NTA essentially as described in EXAMPLE 4. The purified protein has a sequence as set forth in SEQ ID NO:31. Purified huNGFβ-His-GGC is coupled to SMPH derivatized Qb-VLPs according to the protocol described in EXAMPLE 7.

### Example 14

### Efficacy of Vaccination with mNGFβ-His-GGC Coupled to Qβ-Particles that Contain the Polyanionic Macromolecule Polyglutamic Acid

Qβ-particles containing the polyanionic macromolecule polyglutamic acid, herein after referred to as Qβ (PolyGlu), were generated as described in Example 4 of WO 06/037787. mNGFβ-His-GGC was coupled to Qβ (PolyGlu) as previously described in Example 7. The immunogenicity of Qβ (PolyGlu)-mNGFβ-His-GGC was determined in the following way: Female BL6 mice (n=4 per group) were subcutaneously immunized on days 0, 14, 28 and 42 with 50 ug of either Qβ (PolyGlu), Qβ (PolyGlu)-mNGFβ-His-GGC or Qβ-mNGFβ-His-GGC mixed with 0.33% Alhydrogel as adjuvant. At days 0, 14, 28, 42 and 56 blood was taken. Serum was prepared and mNGFβ-specific antibodies determined by ELISA as described in EXAMPLE 8. The titers of anti mNGFβ-specific antibodies induced by immunization with Qβ(PolyGlu)-mNGFβ-His-GGC were significantly lower than those induced with Qβ-mNGFβ-His-GGC and showed a Th2 IgG isotype pattern as indicated by reduced IgG2a but elevated IgG1 titers (Table 1).

TABLE 1
Induction of mNGFβ-specific antibodies after immunization with
Qβ(PolyGlu)-mNGFβ-His-GGC or Qβ-mNGFβ-His-GGC.
Mice were immunized at day 0, 14, 28 and 42 with Qβ(PolyGlu),
Qβ(PolyGlu)-mNGFβ-His-GGC or Qβ-mNGFβ-His-GGC.
Blood was taken at day 0, 14, 28, 42 and 56, serum was generated
and titers of mNGFβ-specific total IgGs, IgG1 isotypes and IgG2a
isotypes in serum samples were analyzed by ELISA.

| | | | Qβ(PolyGlu)- |
|---|---|---|---|
| | Qβ | Qβ-mNGFb- | mNGFb- |
| Days | (polyGlu) | His-GGC | His-GGC |
| | TOTAL IgG | | |
| 0 | 0 | 0 | 0 |
| 14 | 0 | 4063 | 2186 |
| 28 | 0 | 6054 | 1737 |
| 42 | 0 | 5396 | 1448 |
| 56 | 0 | 11703 | 2892 |
| | IgG1 | | |
| 0 | 0 | 0 | 0 |
| 14 | 0 | 1245 | 3804 |
| 28 | 0 | 1952 | 3438 |
| 42 | 0 | 1979 | 2470 |
| 56 | 0 | 4762 | 3789 |
| | IgG2a | | |
| 0 | 0 | 0 | <100 |
| 14 | 0 | 3442 | <100 |
| 28 | 0 | 5247 | <100 |
| 42 | 0 | 5802 | <100 |
| 56 | 0 | 11898 | 111 |

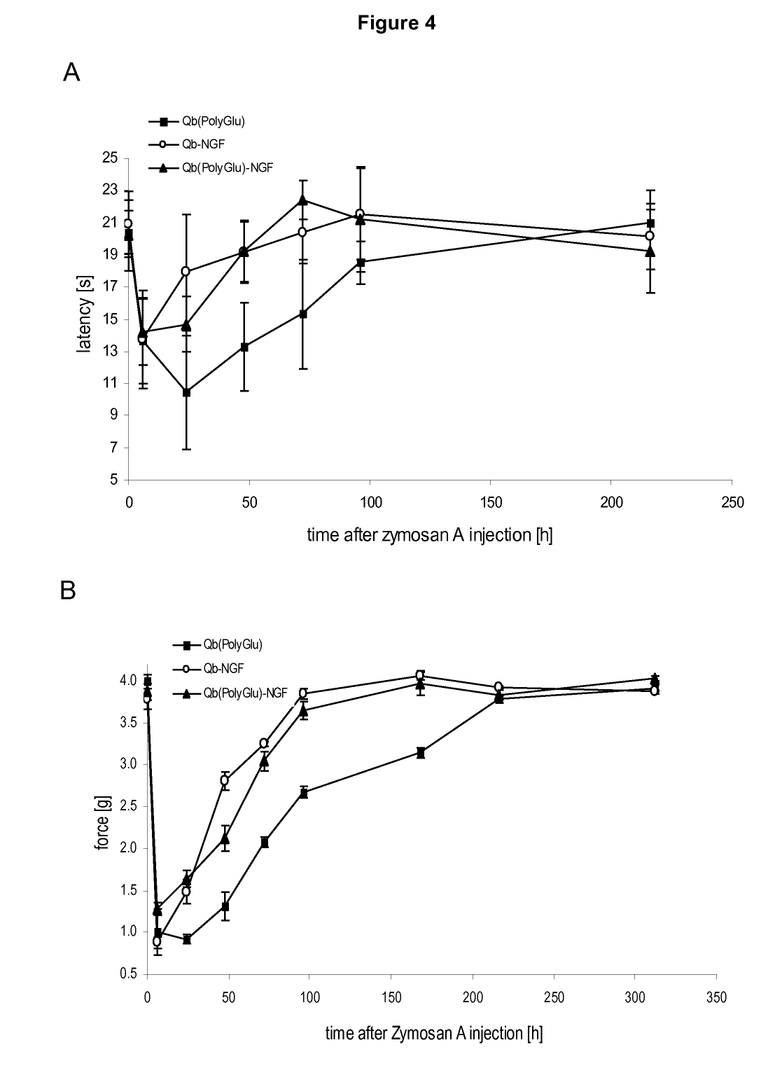

The ability of anti-mNGFβ-specific autoantibodies induced by immunization with Qβ(PolyGlu)-mNGFβ-His-GGC to suppress nociceptive pain in a zymosan A induced inflammatory pain model was assessed in the following way: Female BL6 mice (n=4 per group) were subcutaneously immunized on days 0, 14, 28 and 42 with 50 μg of either Qβ (PolyGlu), Qβ (PolyGlu)-mNGFβ-His-GGC or Qβ-mNGFβ-His-GGC mixed with 0.33% Alhydrogel as adjuvant. On day 62 inflammatory pain was induced by injection of 20 μl of a 3 mg/ml solution of zymosan A into the plantar side of the left hind foot. The ability of the vaccine to reduce hypersensitivity in response to thermal and mechanical stimulation was assessed as described in Example 11. As indicated in FIG. 4 anti-mNGFβ autoantibody levels induced by vaccination with Qβ(PolyGlu)-mNGFβ-His-GGC were sufficient to reduce hypersensitivity after both thermal and mechanical stimulation. The reduction was similar to that observed after immunization with Qβ-mNGFβ-His-GGC.

### Example 15

### Suppression of Neuropathic Pain by Qβ-mNGFβ-His-GGC; Efficacy in a Taxol-Induced Neuropathic Pain Model in Rats

Adult male Sprague-Dawley rats are immunized on days 0, 14 and 28 with 500 μg Qβ-mNGFβ-His-GGC or Qβ alone. On day 35 neuropathic pain is induced by 4 intra-peritoneal injections of 2 mg/kg of paclitaxel on alternate days (35, 37, 39, 41). Beginning three days after the last injection of palitaxel and continuing for 4 consecutive weeks, tests for altered pain sensitivity are performed every other day by measuring the heat-hyperalgesia and mechanical allodynia on the plantar surface of hind paws essentially as described for mice in Example 11.

### Example 16

### Evaluation of NGF Muteins as Possible Antigens Coupled to Qβ-VLP

NGF muteins (SEQ ID NOs 45 to 75) with sequence modifications as described above are recombinantly expressed and purified as described in Examples 1-4. Bioactivity of the produced muteins is tested in the in vitro bioactivity assay essentially as described in Example 4. Muteins with low to no bioactivity in the in vitro assay are selected and coupled to Qβ VLPs essentially as described in Example 7. Immunogenicity of the muteins coupled to Qβ-VLP and neutralizing activity of the induced antibodies are determined essentially as described in Examples 8 and 9. Muteins which show low to no bioactivity but still induce neutralizing antibodies when coupled to Qβ-VLP are selected. In vivo efficacy of induced antibodies to suppress pain and cachexia is tested in in vivo models as described in Examples 10-15.

### Example 17

### Coupling of mNGFβ-His-GGC and Flag Peptide Antigen to AP205 VLP, and Immunization of Mice with mNGFβ-His-GGC Coupled to AP205 VLP

### A. Coupling of mNGFβ-His-GGC Peptide and Flag Peptide to Recombinant AP205 VLP

The peptide mNGFβ-His-GGC (SEQ ID NO:31) and Flag (SEQ ID NO:43) is chemically synthesized according to art-known methods. AP205 VLP (SEQ ID NO:14) is expressed and purified as described in Examples 1 and 2 of PCT/EP2003/007572 (pg. 75-79) and is resolubilized in 20 mM Hepes, 150 mM NaCl, pH 7.4 buffer (HBS buffer). Resolubilized AP205 VLP is then reacted at a concentration of 2 mg/ml (determined in a Bradford assay), with 2.85 mM SMPH (Pierce) for 30 minutes at room temperature (RT). The reaction mixture is then dialyzed against HBS buffer, and reacted with 0.714 mM mNGFβ-His-GGC or FLAG, diluted in the reaction mixture from a 50 mM stock in DMSO. The coupling reaction is left to proceed for 2 hours at 15 ° C., and the reaction mixture dialyzed 2×2 hours against a 1000-fold volume HBS, and flash frozen in liquid nitrogen in aliquots for storage at −80 ° C. until further use. An aliquot is thawed, and coupling of the antigen to an AP205 subunit is assessed by SDS-PAGE and the protein concentration is measured in a Bradford assay. Upon derivatization of AP205 VLP with the cross-linker, dimers, trimers, tetramers, pentamers and hexamers produced by cross-linking, is detected in SDS-PAGE in addition to the monomer form of the subunit.

### B. Immunization of Mice with mNGFβ-His-GGC Peptide Coupled to Recombinant AP205 VLP Analysis of Immune Response and IgG Subtype Determination

AP205 VLP coupled to mNGFβ-His-GGC peptide peptide are injected s.c. in mice (3 mice each) at day 0 and 14. Each mice is immunized with 10 μg of vaccine diluted in PBS to 200 μl. Mice is retroorbitally bled on day 20, and the titer of the antibodies specific for the mNGFβ-His-GGC peptide is measured in an ELISA against mNGFβ-His-GGC peptide. The mNGFβ-His-GGC is coupled to bovine RNAse A using the chemical cross-linker sulfo-SPDP. ELISA plates are coated with coupled RNAse preparations at a concentration of 10 μg/ml. The plates are blocked and then incubated with serially diluted mouse sera. Bound antibodies are detected with enzymatically labeled anti-mouse IgG antibodies specific for the respective subtypes. As a control, preimmune sera of the same mice are also tested.

### Example 18

### Fusion of mNFGβ Fragment to AP205 VLP, and Immunization of Mice with mNFGβ Fragment Fused to AP205 VLP

Constructs containing AP205 fused to the N- or C-terminal part of the mNGFβ 1-10 peptide are obtained by a method disclosed in Example 1 of PCT/EP2005/054721. The method of purification of the expressed fusion protein is substantially the same as disclosed in Example 2 of PCT/EP2005/054721. The construction and sequence of PAP405 and pAP283 are described in PCT/EP2005/054721 and in WO2004/007538A2, respectively. Adult female, C57BL/6 mice (5 per group) were vaccinated with AP205 fused C-terminally to the mNGFβ 1-10 peptide (SEQ ID NO:44). 50 μg of dialyzed vaccine was diluted in PBS to a volume of 200 μl and injected subcutaneously (100 μl on two ventral sides) on days 0, 14, 28 and 42. The vaccine was administered without adjuvant. As a control, a group of mice was injected with PBS or AP205 VLP alone. Mice were bled retro-orbitally on day 0, 14, 28, 42, 56 and 70 and their sera analyzed by ELISA.

1. A composition comprising:
(a) a virus-like particle (VLP) with at least one first attachment site; and
(b) at least one antigen with at least one second attachment site,
wherein said at least one antigen is an NGF protein, an NGF fragment or an NGF mutein;
and wherein (a) and (b) are linked through said at least one first and said at least one second attachment site.

2. (canceled)

3. The composition of claim 1, wherein said at least one antigen is an NGF protein, wherein said NGF protein consists of an amino acid sequence, wherein said amino acid sequence comprises at least 90% sequence identity to the amino acid sequence of SEQ ID NO:22.

4. The composition of claim 1, wherein said at least one antigen is an NGF fragment.

5. The composition of claim 1, wherein said at least one antigen is an NGF mutein, wherein preferably said NGF mutein comprises reduced biological activity, or wherein said NGF mutein does not comprise biological activity.

6. The composition of claim 1, wherein said at least one antigen is an NGF mutein, and wherein said NGF mutein consists of an amino acid sequence selected from any one of SEQ ID NOs 45 to 75.

7. The composition of claim 1, wherein said VLP is a VLP of an RNA-bacteriophage.

8. The composition of claim 1, wherein said VLP comprises recombinant coat proteins, mutants or fragments thereof, of an RNA bacteriophage.

9. The composition of claim 1, wherein said first attachment site is linked to said second attachment site via at least one covalent bond, wherein said covalent bond is a non-peptide bond.

10. (canceled)

11. The composition of claim 1, wherein said first attachment site comprises an amino group of a lysine residue, and wherein said second attachment site is a sulfhydryl group of a cysteine residue.

12. (canceled)

13. (canceled)

14. The composition of claim 1, wherein said VLP is a recombinant VLP, and wherein said recombinant VLP is essentially free of host RNA.

15. (canceled)

16. The composition of claim 14, wherein said composition further comprises at least one polyanionic macromolecule, wherein said polyanionic macromolecule is a polyanionic polypeptide selected from the group consisting of (a) polyglutamic acid; (b) polyaspartic acid; (c) poly(GluAsp); and (d) any chemical modifications of (a) to (c).

17. (canceled)

18. A vaccine composition comprising a therapeutically effective amount of the composition of claim 1.

19. A pharmaceutical composition comprising:
(a) the composition of claim 1; and
(b) a pharmaceutically acceptable carrier.

20. A method of immunization comprising administering the composition of claim 1, the vaccine composition of claim 18, or the pharmaceutical composition of claim 19 to an animal.

21. (canceled)

22. (canceled)

23. (canceled)

24. (canceled)

25. The composition of claim 1, wherein said at least one antigen is an NGF protein, wherein said NGF protein consists of the amino acid sequence of SEQ ID NO:22.

26. The composition of claim 1, wherein said at least one antigen is an NGF fragment, wherein said NGF fragment consists of SEQ ID NO:44.

27. The composition of claim 1, wherein said virus-like particle is a virus-like particle of RNA bacteriophage

28. The composition of claim 1, wherein said VLP comprises recombinant coat proteins of an RNA bacteriophage, wherein said coat proteins consist of SEQ ID NO:1.

29. A method of treating pain in an animal, said method comprising administering the composition of claim 1, the vaccine composition of claim 18, or the pharmaceutical composition of claim 19 to said animal.

30. The method of claim 29, wherein said pain is rheumatoid arthritis pain.


SEQUENCE LISTING (75 sequences)

SEQ ID NO: 1 - protein, 132 aa, Bacteriophage Q-beta
AKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 2 - protein, 329 aa, Bacteriophage Q-beta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAYWTLLIAGGGSGSKPDPVIPDPPIDPPPGTGKYTCPFAIWSLEEVYEPPTKNRPWPIYNAVELQPREFDVALKDLLGNTKWRDWDSRLSYTTFRGCRGNGYIDLDATYLATDQAMRDQKYDIREGKKPGAFGNIERFIYLKSINAYCSLSDIAAYHADGVIVGFWRDPSSGGAIPFDFTKFDKTKCPIQAVIVVPRA

SEQ ID NO: 3 - protein, 129 aa, Bacteriophage R17
ASNFTQFVLVNDGGTGNVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 4 - protein, 130 aa, Bacteriophage fr
MASNFEEFVLVDNGGTGDVKVAPSNFANGVAEWISSNSRSQAYKVTCSVRQSSANNRKYTVKVEVPKVATQVQGGVELPVAAWRSYMNMELTIPVFATNDDCALIVKALQGTFKTGNPIATAIAANSGIY

SEQ ID NO: 5 - protein, 130 aa, Bacteriophage GA
MATLRSFVLVDNGGTGNVTVVPVSNANGVAEWLSNNSRSQAYRVTASYRASGADKRKYAIKLEVPKIVTQVVNGVELPGSAWKAYASIDLTIPIFAATDDVTVISKSLAGLFKVGNPIAEAISSQSGFYA

SEQ ID NO: 6 - protein, 132 aa, Bacteriophage SP
MAKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAY

SEQ ID NO: 7 - protein, 329 aa, Bacteriophage SP
AKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAYWAALLVASSGGGDNPSDPDVPVVPDVKPPDGTGRYKCPFACYRLGSIYEVGKEGSPDIYERGDEVSVTFDYALEDFLGNTNWRNWDQRLSDYDIANRRRCRGNGYIDLDATAMQSDDFVLSGRYGVRKVKFPGAFGSIKYLLNIQGDAWLDLSEVTAYRSYGMVIGFWTDSKSPQLPTDFTQFNSANCPVQTVIIIPS

SEQ ID NO: 8 - protein, 130 aa, Bacteriophage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 9 - protein, 133 aa, Bacteriophage M11
MAKLQAITLSGIGKKGDVTLDLNPRGVNPTNGVAALSEAGAVPALEKRVTISVSQPSRNRKNYKVQVKIQNPTSCTASGTCDPSVTRSAYSDVTFSFTQYSTVEERALVRTELQALLADPMLVNAIDNLNPAY

SEQ ID NO: 10 - protein, 133 aa, Bacteriophage MX1
MAKLQAITLSGIGKNGDVTLNLNPRGVNPTNGVAALSEAGAVPALEKRVTISVSQPSRNRKNYKVQVKIQNPTSCTASGTCDPSVTRSAYADVTFSFTQYSTDEERALVRTELKALLADPMLIDAIDNLNPAY

SEQ ID NO: 11 - protein, 330 aa, Bacteriophage NL95
MAKLNKVTLTGIGKAGNQTLTLTPRGVNPTNGVASLSEAGAVPALEKRVTVSVAQPSRNRKNYKVQIKLQNPTACTKDACDPSVTRSGSRDVTLSFTSYSTERERALIRTELAALLKDDLIVDAIDNLNPAYWAALLAASPGGGNNPYPGVPDSPNVKPPGGTGTYRCPFACYRRGELITEAKDGACALYACGSEALVEFEYALEDFLGNEFWRNWDGRLSKYDIETHRRCRGNGYVDLDASVMQSDEYVLSGAYDVVKMQPPGTFDSPRYYLHLMDGIYVDLAEVTAYRSYGMVIGFWTDSKSPQLPTDFTRFNRHNCPVQTVIVIPSL

SEQ ID NO: 12 - protein, 129 aa, Bacteriophage f2
ASNFTQFVLVNDGGTGNVTVAPSNFANGVAEWISSNSRSQAYKVTCSVRQSSAQNRKYTIKVEVPKVATQTVGGVELPVAAWRSYLNLELTIPIFATNSDCELIVKAMQGLLKDGNPIPSAIAANSGIY

SEQ ID NO: 13 - protein, 128 aa, Bacteriophage PP7
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 14 - protein, 131 aa, bacteriophage AP205
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 15 - protein, 132 aa, Artificial Sequence
  Bacteriophage Qbeta 240 mutant
AKLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 16 - protein, 132 aa, Artificial Sequence
  Bacteriophage Q-beta 243 mutant
AKLETVTLGKIGKDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 17 - protein, 132 aa, Artificial Sequence
  Bacteriophage Q-beta 250 mutant
ARLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 18 - protein, 132 aa, Artificial Sequence
  Bacteriophage Q-beta 251 mutant
AKLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 19 - protein, 132 aa, Artificial Sequence
  Bacteriophage Q-beta 259 mutant
ARLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 20 - protein, 185 aa, Hepatitis B virus
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 21 - protein, 188 aa, Hepatitis B virus
MDIDPYKEFGATVELLSFLPSDFFPSVRDLLDTAAALYRDALESPEHCSPHHTALRQAILCWGDLMTLATWVGTNLEDGGKGGSRDLVVSYVNTNVGLKFRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 22 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 23 - protein, 241 aa, Homo sapiens
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 24 - protein, 120 aa, Mus musculus
SSTHPVFHMGEFSVCDSVSVWVGDKTTATDIKGKEVTVLAEVNINNSVFRQYFFETKCRASNPVESGCRGIDSKHWNSYCTTTHTFVKALTTDEKQAAWRFIRIDTACVCVLSRKATRRG

SEQ ID NO: 25 - protein, 241 aa, Mus musculus
MSMLFYTLITAFLIGVQAEPYTDSNVPEGDSVPEAHWTKLQHSLDTALRRARSAPTAPIAARVTGQTRNITVDPRLFKKRRLHSPRVLFSTQPPPTSSDTLDLDFQAHGTIPFNRTHRSKRSSTHPVFHMGEFSVCDSVSVWVGDKTTATDIKGKEVTVLAEVNINNSVFRQYFFETKCRASNPVESGCRGIDSKHWNSYCTTTHTFVKALTTDEKQAAWRFIRIDTACVCVLSRKATRRG

SEQ ID NO: 26 - DNA, 31 bp, artificial sequence
  Nde-proNGF-F
ggaattccatatggaaccgtacacagatagc

SEQ ID NO: 27 - DNA, 29 bp, artificial sequence
  proNGF-Xho-R
cccgctcgaggcctcttcttgtagccttc

SEQ ID NO: 28 - DNA, 34 bp, artificial sequence
  mproNGFeuk_var1_f
tagattaattaaggaaccgtacacagatagcaat

SEQ ID NO: 29 - DNA, 32 bp, artificial sequence
  mproNGFeuk_var1_r
taggcctcgaggcctcttcttgtagccttcct

SEQ ID NO: 30 - protein, 131 aa, artificial sequence
  Mouse NGF with his tag and GGC linker
SSTHPVFHMGEFSVCDSVSVWVGDKTTATDIKGKEVTVLAEVNINNSVFRQYFFETKCRASNPVESGCRGIDSKHWNSYCTTTHTFVKALTTDEKQAAWRFIRIDTACVCVLSRKATRRGLEHHHHHHGGC

SEQ ID NO: 31 - protein, 131 aa, artificial sequence
  Human NGF with his tag and GGC
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRALEHHHHHHGGC

SEQ ID NO: 32 - protein, 120 aa, Bos taurus
SSSHPVFHRGEFSVCDSISVWVGDKTTATDIKGKEVMVLGEVNINNSVFKQYFFETKCRDPNPVDSGCRGIDAKHWNSYCTTTHTFVKALTMDGKQAAWRFIRIDTACVCVLSRKTGQRA

SEQ ID NO: 33 - protein, 120 aa, Canis familiaris
SSPHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRA

SEQ ID NO: 34 - protein, 120 aa, Equus caballus
SSSHPVFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKTGRKA

SEQ ID NO: 35 - protein, 120 aa, Cavia porcellus
SSTHPVFHMGEFSVCDSVSVWVADKTTATDIKGKEVTVLAEVNVNNNVFKQYFFETKCRDPSPVDSGCRGIDSKHWNSYCTTTHTFVKALTTANKQAAWRFIRIDTACVCVLNRKAARRG

SEQ ID NO: 36 - protein, 120 aa, Pan troglodytes; identical to SEQ ID NO: 22

SEQ ID NO: 37 - protein, 120 aa, Sus scrofa
SSSHPVFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAGRRA

SEQ ID NO: 38 - protein, 120 aa, Gorilla gorilla; identical to SEQ ID NO: 22

SEQ ID NO: 39 - protein, 120 aa, Pongo pygmaeus; identical to SEQ ID NO: 22

SEQ ID NO: 40 - protein, 131 aa, Artificial sequence
  a mutein of the coat protein of AP205
MANKPMQPITSTADKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 41 - protein, 130 aa, bacteriophage AP205
ANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 42 - protein, 130 aa, Artificial sequence
  de Met of SEQ ID NO40 of bacteriophage AP205
ANKPMQPITSTADKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 43 - protein, 11 aa, Artificial sequence
  Flag
CGGDYKDDDDK

SEQ ID NO: 44 - protein, 10 aa, Artificial sequence
  NGF fragment (1-10)
SSSHPIFHRG

SEQ ID NO: 45 - protein, 111 aa, Artificial sequence
  NGF mutein
GEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 46 - protein, 120 aa, Artificial sequence
  NGF mutein
SSSAPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 47 - protein, 120 aa, Artificial sequence
  NGF mutein
SSSDPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 48 - protein, 120 aa, Artificial sequence
  NGF mutein
SSSHPIFARGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 49 - protein, 120 aa, Artificial sequence
  NGF mutein
SSSAPIFARGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 50 - protein, 120 aa, Artificial sequence
  NGF mutein
SSSAPIFAAGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 51 - protein, 120 aa, Artificial sequence
  NGF mutein
SSSHAIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 52 - protein, 120 aa, Artificial sequence
  NGF mutein
SSSHPIAHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 53 - protein, 120 aa, Artificial sequence
  NGF mutein
SSSHPIFHRGEFSVCDSVSVWVGDKTTATDIKGKEVMVLGEVAAANSVFKQYFFETKCRDPNPVDSGCRGIDSKHWNSYCTTTHTFVKALTMDGKQAAWRFIRIDTACVCVLSRKAVRRA

SEQ ID NO: 54 - protein, 120 aa, Artificial sequence
  NGF mutein
SSSHPIFHRGEFSVCDSVSVWVGDKTTATDIKGKEVMVLGEVAINNSVFKQYFFETKCRDPNPVDSGCRGIDSKHWNSYCTTTHTFVKALTMDGKQAAWRFIRIDTACVCVLSRKAVRRA

SEQ ID NO: 55 - protein, 120 aa, Artificial sequence
  NGF mutein
SSSHPIFHRGEFSVCDSVSVWVGDKTTATDIKGKEVMVLGEVNANNSVFKQYFFETKCRDPNPVDSGCRGIDSKHWNSYCTTTHTFVKALTMDGKQAAWRFIRIDTACVCVLSRKAVRRA

SEQ ID NO: 56 - protein, 120 aa, Artificial sequence
  NGF mutein
SSSHPIFHRGEFSVCDSVSVWVGDKTTATDIKGKEVMVLGEVNIANSVFKQYFFETKCRDPNPVDSGCRGIDSKHWNSYCTTTHTFVKALTMDGKQAAWRFIRIDTACVCVLSRKAVRRA

SEQ ID NO: 57 - protein, 120 aa, Artificial sequence
  NGF mutein
SSSHPIFHRGEFSVCDSVSVWVGDKTTATDIKGKEVMVLGEVNINNSAFKQYFFETKCRDPNPVDSGCRGIDSKHWNSYCTTTHTFVKALTMDGKQAAWRFIRIDTACVCVLSRKAVRRA

SEQ ID NO: 58 - protein, 120 aa, Artificial sequence
  NGF mutein
SSSHPIFHRGEFSVCDSVSVWVGDKTTATDIKGKEVMVLGEVNINNSPFKQYFFETKCRDPNPVDSGCRGIDSKHWNSYCTTTHTFVKALTMDGKQAAWRFIRIDTACVCVLSRKAVRRA

SEQ ID NO: 59 - protein, 120 aa, Artificial sequence
  NGF mutein
SSSHPIFHRGEFSVCDSVSVWVGDKTTATDIKGKEVMVLGEVNINNSVAKQYFFETKCRDPNPVDSGCRGIDSKHWNSYCTTTHTFVKALTMDGKQAAWRFIRIDTACVCVLSRKAVRRA

SEQ ID NO: 60 - protein, 120 aa, Artificial sequence
  NGF mutein
SSSHPIFHRGEFSVCDSVSVWVGDKTTATDIKGKEVMVLGEVNINNSAAKQYFFETKCRDPNPVDSGCRGIDSKHWNSYCTTTHTFVKALTMDGKQAAWRFIRIDTACVCVLSRKAVRRA

SEQ ID NO: 61 - protein, 120 aa, Artificial sequence
  NGF mutein
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDAKQAAWRFIRIDTACVCVLSRKAVRRA

SEQ ID NO: 62 - protein, 120 aa, Artificial sequence
  NGF mutein
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGAQAAWRFIRIDTACVCVLSRKAVRRA

SEQ ID NO: 63 - protein, 120 aa, Artificial sequence
  NGF mutein
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKAAAWRFIRIDTACVCVLSRKAVRRA

SEQ ID NO: 64 - protein, 120 aa, Artificial sequence
  NGF mutein
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDAAQAAWRFIRIDTACVCVLSRKAVRRA

SEQ ID NO: 65 - protein, 120 aa, Artificial sequence
  NGF mutein
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGAAAAWRFIRIDTACVCVLSRKAVRRA

SEQ ID NO: 66 - protein, 120 aa, Artificial sequence
  NGF mutein
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDAAAAAWRFIRIDTACVCVLSRKAVRRA

SEQ ID NO: 67 - protein, 120 aa, Artificial sequence
  NGF mutein
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWWFIRIDTACVCVLSRKAVRRA

SEQ ID NO: 68 - protein, 120 aa, Artificial sequence
  NGF mutein
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCTLSRKAVRRA

SEQ ID NO: 69 - protein, 120 aa, Artificial sequence
  NGF mutein
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVASRKAVRRA

SEQ ID NO: 70 - protein, 120 aa, Artificial sequence
  NGF mutein
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSSKAVRRA

SEQ ID NO: 71 - protein, 120 aa, Artificial sequence
  NGF mutein
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRSAVRRA

SEQ ID NO: 72 - protein, 120 aa, Artificial sequence
  NGF mutein
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLTRKAVRRA

SEQ ID NO: 73 - protein, 120 aa, Artificial sequence
  NGF mutein
SSSHPIFHRGEFSVCDSVSVWVGDKTTATDIKGKEVMVLGEVNINNSVFKQYFFETKCRDPNPVDSGCRGIDSKAWNSYCTTTHTFVKALTMDGKQAAWRFIRIDTACVCVLSRKAVRRA

SEQ ID NO: 74 - protein, 120 aa, Artificial sequence
  NGF mutein
SSSHPIFHRGEFSVCDSVSVWVGDKTTATDIKGKEVMVLGEVNINNSVFKQYFFETKCRDPNPVDSGCRGIDSKHWNSYCTTTATFVKALTMDGKQAAWRFIRIDTACVCVLSRKAVRRA

SEQ ID NO: 75 - protein, 120 aa, Artificial sequence
  NGF mutein
SSSHPIFHRGEFSVCDSVSVWVGDKTTATDIKGKEVMVLGEVNINNSVFKQYFFETKCRDPNPVDSGCRGIDSKAWNSYCTTTATFVKALTMDGKQAAWRFIRIDTACVCVLSRKAVRRA